SEQUENCE LISTING (4 sequences)

SEQ ID NO: 1 - DNA, 8980 bp, Mammal
  promoter (1)...(8941): PSP-1
cttgcgttactgtggctgtggtgtaggctggccgctgtagctccagtttgacccctagcctgggaaccgccatgtgctgtgagtgtggccctaaaaagacaaaaaaataaataaataaaataaaataaatcaccatttggattctcctcgaaacttaagttttgttaattcaggcagtcagcatgaatgatattcatacatgtctgggcttttcagaatctactgtgtccatcccctaaacctgctctggagcaagaaaccacagataactctaggcatgttcactagataagactttgtaataaattaattaattaataattttttttaaagacctttgtatttgaatggacgaaaagttaactcaatctaccctttggtctcctgccacctcctgctgtcactgtccttcagagagaatgcagaagaaaaagatttgttcctctggtcattttggataaaaataactatttcagttgttccatggagaactgcgaccgtattagaaagacaatcaggtaacacttctacaaaggtttgtttattaaagaatctttcacattgagaacaatccccaaaatgatctttatggcacagaggcaatatctggtgacaaacatgttgagaactagcccgtttagccagttatagtgtaagtaggaatggtcaaggctctgagaagtcctgcagtgaggaaaaggatttactttcaacatcaactgatgaccttggaattttaaaatgatcatgtaatatcctaaaaatgcttcttttttaatacattggtgccactgaccaactccaaccacagagggtatgtgtcacatttttccacctgaaacacagggcaggggtttcttattcccaggcctgggtgtttgtgcagcagatgcctacaaatgcagagcccccctcaacctgcccgcacaaccccccccaggctgccccaccacatctcactcctgtcccacccaagcctccacggagcccacacaccctcagtctcatcctttcctgcccggtaggggcttttgttctaaataagatcatcttccccttcttacatgtgaaaggcatacatgattcttaaaaaaaataaacttcaagactctgatttcttgggataacgccagggcttcagaggaagatgggcaatatgataaatgggtaagttcactaggaatcatggttttgagttaccaatttacaaccgactgagcctctgggcgtcgtctctctcacctttgctgctgaaaaaattctcagtgtttgacaaaaatctcagttgctagggaaggaagaaagctctggaagagaaaaggggggatggcaagtgacccgcgggtcacatttcctggaagagtccctgtgtccagcggaatctcagctcttcccaacgcttagtccatgtacctaccgaagcctgccttgtgccaggaaacaaccaaacagaacccgattgctgccccctagcagcgtccagtccagtgggagggaccagggcagagaccagtcacctgcatggtgccccatgggggctggagctgcagagagcaggcaggcggggtgggggacgggcaaagagcctgtgggaaccctcctcccccccctcttcccagcaccactgcaggagcttaaggcctgtgtccttcttgctgggactctggtggccttttcccaaatggggtcagggccccaactcgctccctcctgtcaccaggagcctgcagccacatctgaccccctgacctctccatgcccagctctgcccttctgcccacaatgcttcccagccctgctggccaacaagaccctgacatcagctgccccgtggagtctggcctcagtttcctgaaaggccctccctcgcccttccttcttcccctccccccagacagccagctctgggtgctgtcacctgcctggggacccccctttcctcctctgctgggctcattctgatcctgaccttcctctccttcccagaccagccagcggttgaccttgtggaggaggccagcacccaggggagctaccacagtgccaccatctctgctggcgtcttcctggtcatggtcttggccgtagcagccttcgcactggggaggcgcagctgtgctgccagcggccagcctctgatctctaagatgtgatgcagggaagggcgcatggtgcgcagaggctcccagctctggagtccccgggaggaaggaaaaaacagtgtctcctgctccaaggtatctgcaaatatacaccttccttttggaatagctactctattgaaattccaaaggactatcttaaaatgacattccctaggagttcccatcgtggctcagtggttaacagatctgcctggcatccatgaggatgcaggttcaatccctggccttgctcagcgggttaaggatccagcattgccctgagatgtgtaggttgaagatgcggctcaggtcttgtgttgctgtggctgtggtgtaggccggcagctgcttcaatctgaaccctagactggggctggtgcagccctaaaaagaccaaaaagaaagaaaaagtcttattgttttagaatataaataactttcaaacctcagaactgctcaggagctcttggttggcctgagaaaggtggtggcttttcagccagtttgagaaacactcctttgagtttgggtgaagaggctgcagtgagctccttctgggagaatcctcctagcattcaacacataagcatgtggataacacaagagaaaagcaaaaagctcacataggttcaaacaggaaagcagtatttccagaaacaatgacccagagatgccagtgtgttaaagctcaggggtcaagtgtgagttgggagccttcctgtctccccactgacaggacacgctggctaagcagnccgggcctgaagtgcatggacccaaccaatgaccaccagccaggggaggctgatgaaaatcttgtgctggacctggggccaccgctgtgggaggcggaagacacacacagcccaggggctccagagtctctgattagatggaggctagatctgccctttctccagtgtctctgcaggagggtcatttggttcctggctctgagcctgggaatcagccacgtggaggtagctgtaaaaccattaagaccaagagatgagagcctgagggcggaggatgaggacaggcctaggggaggggggataggactaggggagaggagacagacacagagaggcaaaaacatcccactccaaatggatctgtgaaacccagttacaacacagaggtagcaaaatgctaggcactgtggccctcagaatcaacaattggtgcagagatgcactccgggttattttttcaggggaaatttttttaaggaggtgaaatttacacaacgtaaaatgaaccattttaaaatgcatttcagtggaattttgtataatcacaacactgtgcatccttcgcctccatctgcttccagggcattttcatcagccctaaatgaagccctgggcccatcaacggtcctctcttgccttgcggcccccactcggcttccatctctatgcattcaccttgtcactccaggttaattttgaagaacattcctgaaaagactgtaagaagtatcttgaaggcgactcatggaatcattgaaggcataatttgttattttctaaaaaataatttattgtgaaatactttcaaacccacaagaagttgtgagaatcacacaaagaactgtcgccctaaagagatcctcccaattaacaactcactgcatttgctccaccaccgtctctgtctctgcttattcctcttgtcatgagtaattttctggaccatttgagagggagctggagacaaggtgacccatgtctcctgaataaggcggaacaatgatatctttctgcatcgtcgccaattcagaaatcagctctcctaagaccctcctgtccaggccacagagtccactaggatgttaccaatagtcccctgaaggtcccttgcatttttggtccaaaagagccactcagaaacacacagagcatttagctctcatgtatcttacttaagtatcttgcaacctgaaagcttccctagtctttctctgcctgccatgtccttggtagtcttcagcagggtgggacctgacgtctctagaatgtctctccgtccaggttggtctgctgtttcttagccaggtgatcacaggaccgacgctctgctccttctgtgcctcacaccaggggcacctgatgtcaaaccagcccacccatggtgaccttaaccttcattgcctgctcacgttgacagagtctgtgtctctggcggggggttgggggggcatctttccctttgtagttgaatagcattttgtgggaagatattttgagattttgttatgatgctttttctatttgcctctctttcactaactctggctcctactgacaactcctacctgaattcagacaacaggttgtctgaaatcctagacaactaagatggttatgaaatggtgacttttttatttgattgattccttccacagttatttggtggacttttcctctaagggcgagtgtcatctttctgctatcttactcgtttgtattaatttcctatttaatattactaggaagttattgatagctattctagtcaatagctgtgctccattattatcattacatattctgatagtcagtttgtatcatatttgaccatgagggctccttccaggtggttcctgagtccttttaacacgttcccatccctctttgtgtatttccttcttttctggtgcaagaagatgtccaaggtcatcttatactccccctgccacagcccttgggatgagccgtctctccaaggagtcttggttccttgaagtagggtgtgcttttcagaaatcaaattgtggggcttggtgtgctcattgctactgggatcattgcttytaagtcctctcagtgggcggagctgggaaatacacatatgtacatttgtatcttcacatacatacatacatacatatgtgtgcacacatatcactatgataacatgttaatttcaattttaattcaaatattttagttctttctgtcatttccccattcatttttaaaaatatcttaggttcccaaatagcaacatatttgcttactcaagttactaatttgctcaatgcaacagtctcctgacaatgccagctccttcagctgccatcacagtcccacctcattgtcccatgaacttgagccagcaggtgtgctgcctccaggcctcactgctccgttctccttcagcactctgtgtccccaggcactgacggatgcaatgctgaggggcctctgtgggaccccttcctgtcaccccttaccctctgtatctgactcctgcccaccagcaggaaaagagaaggaaaggatgggtaagaaggacaaatgagctgtcacttttattttaagagaaaaaaattaattgcgacacgtcagaaatagagcaggtataataattgaagtaaaactgaaattcatgggatacactctcagctacacaatccaagatcaaattaatgatttatgggaaatttggggggttattatagtgttacagtcaaatataaagaaatttttttaatgaaagagacgtaggctagatcaaaattctataaagacatcaaaattatagacacacacaaagacgaaggggatgaaatgcaggcaagaaatatttgacgagttttggcaagattttctagaacaaatggaaattatgaatcctcacaatcaagatgccctaatgaggaaatgtgggacttcaaggcttctctaaggagataaaactcaaaaagcctccagccaagataagaattaccagacacttatcattccacctgtctcactgtaattgttacaatgtccctaaggacctccccaccccagcaacttggcctactcctccctgccccctactaggaaaggtatgcggcccactccttaccctgccctactggggcaacatacgcccccaacgaaccaagtgtatcccaggaccccatccttccccagccagcaagcgtatcagaagcccccatccttccaaccaatcagcaggtcaacaggtgtatggcaagaccactcctggcactcctttacctttgggctataaaaacagacatgaccatgagcctagggttggctctccctgatgtcaggaagtcggcccactgtgttaagtgtctcttgcctcaataaacttgccttttctacactttgctttgaatccggaattttttttttccacccacatgcagagaccacaacaggacagtgtagggtaaataaaaccaaaggtcctaatacatgtaccgcagcatatctttaaaaagggcagatgaaaagtacatattatattcaaggaaatggtagctaaacaaacagcttctcaggagtgataacaggagtcagaacacaatgaaacaatatcttcaaaggacaagaaaaagtaaccagaacactagaactgcaaatccaacctaagtaagataaccaaagacaaaatgtgctaccacccaccagcttcactaaaacactgagaaacagttcaggaggaggaaaaactaagagggaaagacaaagaaaatataagaatggtgagcataaaaattgagaaaacattttaattaaacattgactgcccaaagaacacttctgttgggttaaacgatctattttaaaaatctattcattgtagcaagatgggggttattcaaggaacagctgttaaattctgtggattttgactcttgtgtgtttgatcctttaagtgtaaaataaatgtacattttattaggtaaaaactaattttagtagtattttttaaactactaaagtaatcactaaaaatgtacaattatctccaaatcattagagcaaaatatggcaaatcagaacactgtatcaagtaaagaggaagcacgaagaggacaaaaaaaatttaatgagaagatgcaagaaaaaccttataaaagtgtagaaataagtcaaaatatgtatgtgaccatactaaaaataagtggattacatgaaagtagagatttaagcatataattcaatattaaaatataaaatttaaatgccgattattatatgtcaatgctgggcatgcaaatgggyataatcagtttggaaaaccgataaaaggaattctacttctcactagaagtataaatgtttttatctgatccctcaaagaaaagtaggagaattcttagagcagcaccgctgagcagaatcctkgaactacctaaatttctacccagagaaaaatggtgggaaagaaattgttgtacatcgacctacaggagtactaatcaggagttcctgctgtggcgcagaggaaacaaatccgactaggaaccatgargatgagggttcagtcctgggccttgctcagtgggttaaggatccggcgttgctgtgagctgtggtgtaggtcgcagactcaactcagatccttcattgctgcggctgtgatgtaggcaggcagccgtagctctgatctgacacctagcctgggaacctccatatgctgcaattgcaggcctaaaaagcaaaaacaaagaatactatccaacagtgaaaacccatgtgctacacaaaaacagcattaacgaatctcagaaacttcatattgaaggaaagaagccagacataaagaaggacatccttgaagctttcatgcatctagagtcccaaacggacagggctcattcataccgttggaagcctggaaaagggttagccttggagaagctggttagtgacagaaattcgtcattacaagaattcttggatgctgcaaataagccagacacttacatactacttttagcacattgggctcagaaggagttttggaagatttggaaaacttcagagttgtttaatgtcagtagaatgggctctctggtctcctggttggtgtgctggtggtgacggtccttgaaggcagttgggtggccatggctttttatggcaggatgacttccgttctatagacagaccaggtgttagttaccccttcatcagtggctggacatttgagtctttcctgttctttttctcttatgaataatgctgctatgaatagtcacggaaaattttctatgtggtcatacattttcatttccttgggtaaataaatatggagtgaaagctgagccatgtggaaaatcatgtgaaatgtgcggagaacctgctaaactgtcttgcacttgcaggtctgctgggttgtcaaaactgggatgctctcactggactgagccgtgtggtcttggtatcttaaatatacaacatatagaaacacctgcacacctaacgtccttgtggacacgcccacacacaggtggtgtgcactgcagctgccccacgggtgctgacccacacctggagcagacggggggctcttcgtgtgactttgccctgagggacatgggacagctctgagaccgggcagccaggagctcagactctgtcctggttctaccactgtcaccctgtgactckgggcaagtgccttggtctctgtgggtctcagttcctgtcctgtccagggtggtgtgagactcatgcaggttcacggaggtgaaagtgcagtaacttaaccccaggcagggcggctccccaacaggaggtcctggtgcttgtggccaatctgttacccttcatccaggatgacagaaaggggacaagaagagaagaaagcaggcgtcgtatcccagtctaggtggggaaagacccagagagggtctgagaaaggccagggtgctgagcaggcacccctccctctgagcaagccaaggcccccagcacctcagcccctgcagccctgctggcctgggccccagggggagggaggagatcaggttgcagcggctgggctatatagccccagcctccagggccaggctc

SEQ ID NO: 2 - DNA, 13116 bp, Mammal
  promoter (1)...(13081): PSP-2
cttaacttgctgctgaacaccaagattcttttttctttttctttacagccataccagcagcatatggaagttccctggtgggccagaggtagaacaggagctgccattgccggcctacaccacagccacaacaacgtgggatctgagccaaacsgcatctgcaacctacaccacagcttgcaggaatgccagatccttaacctaagcaaagccagggctcaaacccacatcctcatggaccccatgttgtgctcttaacccactgagtcacaatggaggaaactctgagacttttttttttttttttttttttaagaatttagaagcttgttctatggccttggccttggagttattcagaaagttttattcctccccaaattatataacttgcactatatgccctgtgtctttttacaacttttgaccttgtattcatcaaaaaaacctccaccttacccacaaattcattagtttgtataaactttgtccatatcattggttgactgggaaagatgccctacaaaagccaacctagcaccggggcttgatctttctacgtcggctgctcccaccaagcccatctctttcccaccagtatccccaaattgctctaaactttcatctgatgatgcctggcaagccaaggacctcttgtacctccctgggattcttagaatagacttccttggtaagtactggtgcaatgatcaagtcatctgccatctggatggattagaaaaacaaaaagaatggactttggatcagcagagaacctatgtgactagaaaacctgttcatcccaattttaacacaaatgtcacagaggatgaactcagtttccactaagcgaacggtcattcctaccagctgtacagccagatgatgactgtgttaaaattaggccacctgtatttgaaatcatctaatttttccacgtggagtagatgcaatattacattgtaatatcaccaagaacaaacaagacaaaaatggagtggacctatctgaaaactgatgaactgaagtatagaaggaaaaaaataggtgtataaaatgttttccatgagaaaccaagacccttacacagttccaacatgcctgttcacttgcccagcaccttcccaacccttctctactggctactgttttaaagtttgcccttccccacatctggatttttattacagatctaaagctctttcaattttatactgattaaatcagtactgcagtatttgattaaaaaaaaaaattaggccacctgtccaccagcacctgaagcttgaatgggttcctctggttcttctgggaaaatggtatcagaaacctgtaggacctcaaaggctagggagagtcaagacgttgggagagggcagaagagcaccccatggctgtgggtaaatcttgaaatcaattcatagtgctattatcctaggcactattgactaggcattatctaattcaattctgataagtacctgatgaggcagttacttttacgtatgtattatgtatatagttatttattcattcacttattcatttctctatttctctcacaagaggaggacagtaaccccaaagtacaattgcatgttaattccagataaattatccatatttctgccacttcccaccttctgcctggggggcttgatcacaaacactacatttcgacatgtgtccctggaatccttatgttgagcaatgtctgatgtatgaagaagttacctttaaagaatgacataattactggtttcagacccaattctgagagcatgccccctggaaagacagtaacatccacttgttatttctccctaacaagtgttcctgccataatgtcccaatatcacagtgggttgtaataaaagacaagcaacacctactacagtcattgtgataagataaagtcacctgctatcccaagcaacctgctccccaaaggctctagagtattttaacatttgaagaagaaaaaaaaaatccttgttctatgaggcttggcagtctctgctcctctctgctggctgagcaaggccaacagtctgaagtatggactgccttggagattctggtgaccaggagctggtaaaggggttttacaaattatttaggtaatacagatgtgaattatttgatatccatggaaggaataaatggatggtggaatcattcattaacttttttcatcattcatcatccagcaaatatcttccatcttcaagatttttgttcagatcaaaaatcttggtgtcattcttaaatctttctttcatatcacctatctcccctcttcttaccatgtccactgatattaccatgatccaactcaaaatcatatcctttccagagtgacacaatagcctcctaacagttctttggctatctgataacatctcatacagctttctatcaaaaagaaaaacaacccaatcaaaacatggccagaagatctaaatagacatttctccaaagaagacatacagatggccaaaaaacacatgaaggatgctcaacaggctttcccattgtggctgagtggtttaagaacctgactagtatccatgagaatgcacatgcgatccctggctttgctcactgggttgcaaatccggtgttgctgtgagctgcagcaaagtaatagatgcagctcagatctgacgtggctgcggcacaagctggaagttgcaggtccaattcaacccctagcctgggaacttctatacgctgcagagtgaccctaacaagaacaaggagaaaaaaaaaaaaaaaaaaggaactaacatatgatccagcaattccactcatgggcgtatacccagagaaaactttaatttgaaaagatacatacaccccagtgctccttgcagcactatttmcaatacccaagacatggaagcagcctaaatgtccatccacagaggattagataaagagtatctggtacatacacacaatggacagttacttgaatgaaataatgctatttgcagcaacatakatggacctcgagagtatcatactaaatgaagtaagtcagagaaagaaaagtacggtatgagagcactaatatgtggaatctaatgaaatcatgcagaagaactgattcacaagacagaaacagactcaaagattttgaaaccaaatttatggtcacccacggggaaatgttagaggaaggggtggatggggaggttgggattagcatatacacactactagatgcataatcagtaacaaggacatagttaacatagggaaatctattcaatactctgtgatagtctccatggggaaataatctgaaaaacaaaggacgtatatgtataggtatagctaattaaatttactgtaaccttaaactaacacaacattgtatatcacctacactccaataaaatttattttaaaaaagacataacacagtgagatagtggttatgttaaaatacattacatcatgtcgctgcattattcaaagccttccaagagcttactcaagaagaaataggaagacaacaaccagagactagggattctggctataggaatttcaagatcctggaacctctggaactgaaactgtataagcccctaagtccttgtgttttgaagtaaaaggcttttccctgagtgtcccagcttgccctccgtctcaaaacgctgactcaagagttaatgattaggaaacgttgttacagtcaaaactgggtaaaacaagtctccatggtgttatgccctgccttggcccccaagctggtctctaaccagctttacacaagtgatttcttccttgcttaactgctttcttactaatggttaatttctacttagagttctattgcactgggtattttcttttcctctgaatcactccctcacaccggcttcctttggtgagcaatcttcattgcagagagaaggtcaaggtgcattgaccccaaaagaccatcagacaccatcaccagaccatgtgactccagccttgatgactttgaaatgatctacggargcagagatggcaggcaccctaatccttatgaacaggccctgctttttttttttttttttttttttcaagctacgcctataaaacagcttgctatccattctcaagggaggacgccttttgaggcataagcctgcggtggccccctttgcctggcaaagcaataaagctgttctttctgcttcactcaaaattctgtctctgagacttaattagtggtggtgcacagaggccaggtgtcagctacagaagaactctatgttaattgttctcggatctgatgtgggtttagaaaagaaagaggattgtggaaaggctttgtccagctcctccttggtcctgtgaagcaccctaagattggaaggtgttggcagatgtgacaagaaatgagttccggcacccagagaaggtaggaggtggtaccaggcattaggtagaatcaacaactgacccttccctacatgacccctgactagggacctaatgatctcacttccagtttaatgattgttggtgttccagaaattttcggaacttctgcaaatgattggaatgggcttcattgatcttggcatcattcgttgtatcctgggaaggttagggtaagtaaggaaaagnnatccagtgatgagattcagggaaaaaagaactgttgggnaaatggtnaggagttagttattcccttcgcaaagaatgaaccaggcggactaagaatgctgcctgccatatcagtaaacaaaggatgctgtggccattaagcctttaaacctctaaggctgccctgaaggggagccctgatggaattaagaatagaaacagaataccgtccatcaaacagtcaatcactggagctgcccccaccgtgtatgntgaagggattcaggatgggaaagaacccattantggccctagaaanntgaggtgcatgtccaaggaatactttcaataagcccagattctcacatcctcccaagcacagaaaagtgattttgcaaggggtggaaraagtttgagattaacgtatacacactagcatatataaaataggcaaacaacagggacctacttctatagcactgggaaatatactcagtatcttataataatctagaatggaaaagaatctgaawaataatatgtatgtatgtccccccctggtgccacagcaggaactcccaytcttttttaatgtttgtgtttattttaaatgaaaacaacaatgtattgcagtatatttgactgatgatagttgaccaataattgatattatatagtttcaggtataatataaggatccaaaatttatatagattacactccatttaaagttattacaaaacaatggctctattccttctgctgtaccacacttctttatagcttaattattttgtccatagttgtttgtgcctcttaatctcttaccactctcttgcccctcttcctttccctgtgcccactggtaactactagtttgttctcttatctgtgagtttgtttctgttttgttatattcattactttgttttatttttcggattccacatataagggataacatacagtattagtctttctctgtctgacttatttcaccaggcataatactttcaggtccacccatgtcgtaaatggcaaaatttcattctttttgatggctgtgtagtattttacatgttacacttttattaaagttgttgcccttatctattttactactacattatcagcaccttgaggtcaacgggctgacctacgctcatgggtcttcctttcctgcagctccacaccaccagcaaccaagcacactaagaagaaacaagcaaagagaaggtgcatagtaaatgttcactgaatgaacaaatgaggggtgatggagaggaagtgtccttcctctgaggctcatcttgcaaagctctcagatgtgtctgtggaatgaaacacatatggagtttcaaaaatacttggaggctaacatttttttaaaaaaaaataggtcattctgctacataattacatttacagttctcttgaaatctcagatctggccacaccagatcttcctgcccacaaggaaataatcagccagagctcagtcagttgctgtccgcttggctgggtcacacatgcacccgagcaccatggagcagaagcaagcccaccccactcacaccgtgtatttacatcagctgtttgccaccagtagaaatatgaatttctacctctgccctggacaaaaatctgacaggagttcaattgaagtcttctaaatttatctgccttttttttttttttttttttttttttttttttggccttacctacagtatgtggaagttccctggccaagaatcaaacctgcactacagcagtgacccaagccactgcagtgacaacactagatccttaatccacggcaccaaaagagaactcttttttaaaaaatctcctgcctctgtcaggttttctcctcgtcatgacatttttgcaatacattattgatttcctgtttgtccctagacaaggttaatttgccctttcagttgttcttgttttggaaatgtcagttttccacagagcaaagggccattttatgtaaacaaacaacaaaaaaagagtttattcaggaatagcagaggaatcgcaattcagaacaagcaaactatggcaaaacataggcaagtccagaaaataaaagaggagaacattcttttataggggaaaggagaacgttgttttgacccacaactctttggaggaaactgagagcctgaggtggtggtggttcttattggccacagacaaaggcagtctgctggatagaattggaaatcttcctgctgaactatgcaagctgaaaggagtagcatgtgcaggaaaacactgccttcacagcatcccagctcgagttttgagttaggtttccttaaaacattttcacaccaacggcaagcaagttcaatgactatgacagtagagtgactgcatttcctctctgaagcaattagtccaatccagacctcctgaattcaatgttcactgcatatcttccaagaaaataagttgaaaaatcaggtcttgtaggggtttctcttgkggcycaatgggttaagaacctgactagtatccatgaggatgcaggttcaatacctgcccttgttcagtgggttagggatcccacgttgcccaagctgcagcctaaatgtggcttggatcccaagttgctgtagttgtggtataggtccacagctacagctcctatttgacccctaaattccctagcctgggaatttytatatgccacaggagcaaccctaaaaaaaaaaaaaaaaacaaggcctagctcatcagattctgtggactaactgtgaaccagcaaaacatcttgtacttgaaaaataactacaaaagctggaaagctggacacaggtgtctatgctatttacccaatcatacttggcctgggagttccctggtggtctagtggttaagactcagtgctttcaccgcagcatcccagatccaatccctggtctgggagtagagatcccactttgagctgctacttgcttcagaaaaaaaaaaaaaaggagaagaggagaaaagaaaggaaaggaaaagaaaagaaaagaaaagaaaaatcatacttggccagatgaaaagggagcttgctctcacatgtggtaacttgagattttcagaatcggagcaagagaggtgcttgtagaatctctgcagaagctgtgatgttgcaggtccttacataggtcagactcatgctgttgcaaacataatttggaaatgtgcaaaatgtatacttagccctgccctcctcacaccccatatctctcctcctccactcttcatgcttctccctcagggatctcagggtccttatgcttcccatctgctaaacctgtgcaaagttacacattcctccccagaacagtcacagtccctcggaatagcttatcgggattattaaggaaagagtgcacgagttagtggaaataagggcagtgaaattaccaacagaaggggaaaacttcaggaatgaaacactgtcgcttctcagtggaatcccccttacagatacggagatgcacctaagggcctcagagaaatcagagatgctgctggaggctcggaccgttacacaggatgatttagagtctccgtcctcctagctcttctcatttccttaatgtttcttggtcctttatctagacaaatgttccagccaaaatgcttgccttcaactgtgccaggatgcccaggacattggagcaacccctgagtgaaaaagcctgaaggagttccctggtggctcagtgggttaaggacccagcattgttactgctgtgatgctggctctatcccatgccatggccaaagcaaaaacgaaacaaaacaaaacaaacacacaaacaaaaaaccccgactccatgcaggtgaagagatgggggagtgtgaaactgaacctctattgtaatatagtcagaggttttcgatatattctagtctcacgaaatctacatgaggtagacaggtgagatgatattttccacccctctttgtacatacgtggaaactcagggtcagagaggtgaggcggcttcaagatcacatagccactgggtggcgtaatgaagactttaagacacactttagagatccaaagttcactggttctttmagcaaagtggctcatcctaagattttgcttctctttccctctcctgtaggtttacaatccatcttgtgatgaggttataaaaaatctcaccctcatctcatcagatccataaaggtcaagcatgcagatcaaattctttacgtgattgctcagaaactgcagcaaccggcttcctctctgctacattcaaagacttcaggagttcccttgtggtatagcaggttaaggacccagcactgtcactgctgtggcgtgagttcttttaaaagctgcagactcagccacaaaaaaaaaaaaaaaaaaaatcctcagtgccttaaaatgaccaaggtgacttctctcttatgcttggtggctttggcagtcagatggagcctctattccatgtgtccccattctgaggccatttcaaatgtagctgctcaccatggcagagagaaaaccaaaagcatggtgaattgtgcactgatttctaaggctttgtactcagaaatgacatgcattgcttctgcattcatttcctaggccaaagcatgtcacataaccacacctaacttcaaaagggggcaggaatatgatcaggcaatctcactcctgggcatgtatccagacaaaactgtaattcgaaaaggtacatgcaacctaatgttcagagcagcacaatttacaatagccaagatatggaagaaactaaaggtctatcaacacgtgaatggcgaaagaaggcatggtataggagttcctgttgtggcccagtggtaacaaacctgactaggatccttaaggatgcaggtttgatcagatagagtaggaagttcccaatgtggctcggtgcgttaagaacttgattaatatccatgaggatgtgggtttgatccctggccttgctcagtgggttaaggatcgagagttgccacaagctacagtgtaggtcacagatgaggctcagatccagtactgctgtggctgtgatgtaggctggcaggtgcaactctgatttgacccctagccctgggaaatgcattatgccctaaaaagaaaaaattaaaaaaaaaatagaataggacagaactttgtaacagaatttggtagtataaaaatattcttaaaaagtaagaatttatactaaggatgaaaagataaatcaaaaatcagaaaatctattaaagcaatttctcagattgatagaataagcactgaaattcatattaacatctcaaaggatacaaaaaagattcgattgaacccgactgaattgctgttgatgatgtaaacaaactctttgacactggtcatgggaggaaacaagtttaatgtcataaactcaactattaaaaatactccagcaaaacatctttcctgttgagatttcagaagcgatccgttgatgtcaagagcaggaccgaatggctatcaccagtcgtaaacactggtattggaggctttggccatttagccctttctccacagaaaagcaaagctgtagcaggtcccctcagcttgtggagctggaacacaaaatagactcttcttgttctttagcaacttagaagcactttctgtcctgggattcctggccctggaaggggggatgtttgtgctgcagtcactcaaacccccacaaacaccttaaaccaggagcacagggaggactcctggggcgggaaggggggtttcggggtggcatcgcccagggactctgcactggcagccccgcctgcttctggatccgcctccctggggggcttcccatcaggtgggggacctgggggcacagcactccccctcccctgcagccacacctcccctgtcccttcccagcccatgggggggacacgcccattcctacagtgtcacccaccaggacaggagcctcctgcggcaaccacccctccaccaccacggaggccccttcccagagctgggtctgggctcagcgtctccaaggcccaggggacacgcagggggaggagcgcctgggaaaatggcgggtctctggcaacgaggagggtcctggctggggggcagctcccaactgccccacagaccctgcgcctcttccctgagccccaagcccgggctggaggcagaggatgctgtccacgggcggtctagacagagggcagctggcaggttgtgatggggtggagggcgctggggggcctgggggccaggacggggccccccccagctgyacggagaaggagaggagagccctgcagggggcgccacagaggaggggctctggaagggaccaggctggtttggggccctcagaaaccagctgggacacttggggacaaagatctgagggacacaactgctctaaagctgctgttaacccagacatttcctcaggagggtgtggtggcttcaggaccctcagacgttgattcccgagtggcccctggaaaagggactgtcctttcggtttctccccaggcagcccccgcttccccacctggacgctgctggggggacagcggccctgacacactggagctgaaggcaggctccttgggctgggggggtgagatgaggagctgcctccccccgtggtccccgcctctgctccatccgggcatttacgtccacggggagctcccgtgtcctctccccagaccagggcagggggtgttctgagggcagggagtgtgaggggcatctgggaaggtcaggaaagggagtctggatggaggagaatggggtggcattattgattttattgtagcaataatcagggggctcaagggccgaagtggaattgaccttgagaaggaggacacctcccaagcggcctcccctgcggtcatcaggcaggaggcctgagggagcaggtggctgagggcggcctccctggttctgcaggaggtgagaggctgctctaccctggggacctgagggcccctgtaagagagaagtgggagggagaggacagaaagtcctcccctgtgccttagagaaaggtgagtgacgtaagtgtaatagaaaatcaaccaaaatatgctcgaacatgaatttcaaaaagagcttcatctggactaaaaacaaaaagaggcaagacccctggcaactgggaaaatacacattttaaaagttaggtattagagcttacatatttatcagtaaggaaatttgtaaattcagtaaaatatagaattccttaatttcaggaaaatgggtatgaatatgttttggggcctctgcttggtaggcagttcctggtaggtagttgggctgccatttctttttatggccgaatgatttccattgcatggacagactttgttttaattatccattcatcagtggttggaaatgtgtgtttttaaagtgtttggatcttatgagtactgctgctgngaatatttatgcacaacttcccgtgtggatgtacatttttatttacctgggtaaacacctactagaaatctgagccatatggatactctttggaaccagttgagaaaatgcacatatgtcttctacggggccttctgctgagttattataacaaggacactgcccttggactgaatcccatggtcttggaatcagaatatacaatatatagagacaagtacacacctggcatccttgtggacacacccacacacaggggtgtgcgctgccactgccctgccactgctgacccacgcatggagcagactgggggctctttatgtgactttgtcctgagtgacacaggatggatctcagagacaccaggcagccaggagctcagacctcgtcctgcttcctacactgtcaccctgtgactctgggcacattccttggtctcccgctctccgttccactttcctggaaggggttgtgagactcatacacgttcatggagatgaaagtagcctaaccccaggcaggagggctccccatcaggagttccacgtcatcggtgattattctgtcacccttcatcaaggaccagggaatgcagaaaggaaaagaagaaactggaattcatatcacggctcgggagggttaagaacccaacatagtgtctgtaaggatgagggttcgatccctggcctcgctcagtgagttaaggatctggtgttgcaacaagctgtggggtaggttattgatgtggctcagatccggtgttgccatggctgtgccgtaggctggcagctgtagctctgattaaacccctagcctgggaacttccatgagccgcaggtgcagctggaaaaaaaagagagagaaaaaaaaaaagagaaggtcctagaaaggccagggtgacgagcaggcacccctccctctgagcaagccaaggcccccagcacctcagcccctgcagccctgctggcctgggccccagggggagggaggagatcaggttgcagcggctgggctatatagccccagcctccagggccaggctc

SEQ ID NO: 3 - DNA, 15572 bp, Mammal
  promoter (1)...(15541): AQN-3
gatcatagcaggtgtttagagcctcccaggactggttctggcggtcctctttcttcctggaataaagaatgcttcatcatttgttgggggttttagttctgcagaaagactcaaagatattgttatgtctattccttaaggaggaaccgggaccctaccccaaggctgcactattgtctcttgacttctcttccctgrtcttttcattccttcgcttcctggattacagctaccctttcaagctcagggaagatcctggaggctgaaattcatttcctcaaaacaagaagtggaggacacagaaaggcttgtgtgcccagtgctaggtttgtcccaggaaaacaagacttagctctaggctctcgcccggcttctggagagacatgacgaagacatgactaaatcttccctaaaatcatgtgacccaatcttaaagactaccccactatccaggagaatgtctaaacgtctgttcatatcctactaacaattcccccaaactggtctgttccttttcaccctatttacataagaaatggccaatcagcttctgcrcagaaaccccctttcctggcacccacatataacctycctccttgaccagctgactgctgacatccccttgtgagtcagcccggcagtacaccctagtaataaagttttttatgcctgaacacaggtcttgtccttttggccaaccttacacccaggagcctcccagggctccgcttggttataatattagctgtggttttttttataggtgtccttaattagagacaattcgtggtgtttttttgtttgtttttgtttttaatcatgaatggatttgtattttgacaaatgctttctccggatctattaaaatggagtatttgttctttagcttgttaatataataaatgacattaattgattttcaaatgctgaaccaggcttgtgttctcaggaaaaactcagttggtcgtgacttacaatcttttaaaaagatggttggaattcatttataacaattttgttcaaggatttttatatttatggtcatgaggaatatttttctgtaatacctgggaactgaagtttggcagctgtagatggggctttgtactctctgtagtgaatctgacttgctaggctgtgagcaggagagtggggtaagtagtacaaaacacatggtgacctatgaacttcttggatgggaacagtgaagacagcctgaaccacccaacagagacattcagccatatacgtaagcactctcatgcatatttgtgaaatgtattacacatgatatgtagatgtggatatgggaaaaaactagaaaagaatacctgtctcctttcactactctatattcattagaaaaaaaatattggataaactattaacaagtgcctcaggattaccctgtcagtattgcaatgtgtggggaaaggactttgggtaggatgcaggacctaggtccctgcaatggcactggattgaatatatagggcctaccccttcaaaatggagcttaatctatgattattatatttttctgtgatcacagtcttacttcagaggtaagaagggtaagcaaagctctttgccattgcattgaaaaccagatcataataataactctgctattgaaccagcaagcaattataaggattcttttttacagcatcatttcaaatgtggaatattggaaagaatctaagtgtccaactgttgggaacttggtaagcaaacaatggcaaattcatacgatagactgtttttcagctataaaaatgatgctgtggatggggaggcaataacttggaaagatattaaaatatattattaagtgaagagagcaagttatacaacatctatgtttgattggattttttctaggaaatacttacctagtcacttagctgccatcagttaccacctactgaaggatatagtccttgagtgtataaactggttatctcagtatagaaagattatgactcctttgtgttttatcctcctttgcttacctttatttcaatggtattttttttctttgctgactatcacttcttaaaataagagaaataaatggaatatactgcaatgagacagagagtaagagtgggacaggtagagtgccagcatgctagagagagtgtgtctgggagagaagaaagagtgcattaactcagagatgaactggtgtctccataccactgctgaatctaactctgaactcgacatttgacttcaatccacttcctacactaaataaattcaaaccatgttcctgatgaatgactagtcctttagtttatgaggtgtctccctacagctcatacctgtaacgtgacatcaactcatgcctgtgtctgagctccacttggaccccacctccctccatccatgcagtcatgatataagaaggttttgttttaagtcagaggttggaccatagataatgtaccaggctgcatcaattttatgataggttaggactgttcacaatacacataactacatcatgcagcaaacttccagtctataatggcagtacaagctaattgccagctgtcattcattaatgctcttaacttttctttttttgacactcaggtcaattttctcaattatattacctgctctgacatatattatataaacatgaatataaatttttttgtatattgaaacatttaagtaaaattcagtaactgagattttaatattttgggaattgacttttaaatttataattttacttcttaatgttaaatgtcatacattcaaaattaaagctggttttccaaattcttttcatttattgaagcgtttcatttgttcattctatgtcaggataatgaaaaacaaaatttaactcacccggttcagctaaagagtttaatgaagggatggcgctatgggctgaatggttgggccactcgaaaattcacgtgttgaaacgctactcctcaaggttacagcaaaaacagaacaaaacaaacaagtcatctaggaagtgggctctcaccacagaatcttctgatgccctgcacttggattccccagcctccggaactgtgagaaataacttcctgtcatttaagccatccactttgcggtgttttgctatagaagcccaaacgcaaatggtttactgagatgcaggcagagtcgaagaaaccaaataggatgtggagaccgccaggaactaacaatgcaggaagctgttattagcctcagacctaaaggagcaagaggaggaaataatatttctggaacccagtgagagctggtgtcacagaggagggggcgcctccctggagctgtggctgtgagggcagagccacaccggaggcaaggggccgaaggagaggtacttaatctctctttccaccttcccacctcctgttgtcggtagccagtggacagtcatgcagcaaaaacatctgggagatgcagtctacaagggtcaggacagagaccagcagaagagaatggagagcagagaatgaatgaccgggttgggggggtgagaatagtgtattcacccaagaaatattttgttaaactaattgctatgttccagacattgggctaagtgtacttgacccaataagctttgtttggaatttgtaagtagatctattggggatagatcttgaggcaatttctttatctcttgaatattatttctacttacggcatttcagtagggagggcatttcagtagggatagctccatgtggattaggtttccagtgaggtttgctaccttttgcttctgcttacataataacacactttgaaaagccttaagtataaaacaggcacaacttcttctaattaagccgagaagtaaaatatacagttgaattttggaatatgacatgagggagttttctaggctctttgttggctttaaagaaaaagcacaaataaggatgcagaacacaggagacaggaggcaggaacaaaaacaaataacttggtgagcaattcaaaggggaattcacaaattctgcttcctgaagccttaatgagaaagatggagacatttgggtgaggagttcatattattaccgggaatgttttgtatggagaaatgaaatggattttcaaagaagcagcattctgataaaaatcctaaatcagaaatgaaaaaacttgcttcttcattcagagctacccaagccagtctgtggaatattgaatgaacttgcatttcgctcttctaccccctggcaattttagaggccattcctttgctctttaagcactcctaccgtctcccttcccttcagaggggccaagcggaagaatttagatagtacacgtatatcttatatcacacagtagattggttccttcgccactttattaaattggtatttctttatgaatgtgtaaagcttgagaagcagaatgtccagtagtagcttctgctactgtgtagacttgggcaagccacttaatttctcagaaactcatggtgcccatcggtgaaatgggggtgatggatgaagatcaactggaaaacagagggtttacctgggcttcaagaggtcattaatcacatgaatcatatggaaaattgtgtctgcacatatctgtgtgttcatattcccacttgtgaagacaggatcagtgtgttttactagattctccaagaactctgttgttcctaaaaactattatgcaaatctcaaccaaaacatgttagataaatcctattttaaactgaaaaattctacaactacataaaaatcttccatcgagattgtaatttcagagctgctttgagtccttcctttgagtttcaattcacttgaactgggtttaccaattccaggtttcacttcccacttttttttctttttttttttttttttctctttttgctttttcttgagccactcctacggcatatgaaggttcccaggctaggggtctaattggagctgtagccactggcctacaccacagccacagcaacgcgggatccgagccacatctgtgacccacaccacagctcacggcaatgccagatccttaacccactgagcaaggccagggatcaaacccacaacgccatggttcctagtcggatttgttaaccactgtgccacgatgggaactctgaagtttccactttctttctagttttcatgatctataaacacataaatcagattcctaacaaggacataagccatcaaatacattagaaacgtgcacatttaaaagattatttttatgacataaatagatatgctctattttatctgttttaaaaatcaccattttaatatttggtttctcataacattcaaggaactgcagcttgagatatttaatagaatttcagagtgacagagatcttgcagcagcattgtccaaaacagcattctgagtaaatggaaatattctatgaatttgtattgtatggacagtaaccactagtcataaatttgagcacttgaaaggtggccagtgcaactgaggaatccaaaattaaattaaatttaatttaatcacacgtggcaagagactactatattggattacacagtcttagagattgtcccatacaacccatatcaggagctgagactctcataacacctcctaaaacagatgttctcaacaacagtgctattgagcaaagccaggtgattctttgtgaggtgtgggcagaggggctctcctgtgcattgtagggtgtttagcatcgtccctggcctctacacaatggctatcattagcatcccaactctagtgacaatgacttcagatactgcctggaaggtatatctgccccagttgagaactactgtctggagattttccttgaaaatacccaggggccagacacttcaccagatagagcattccttcttggcacttgaaattattaggaggttattccatacatatgcccgcccatacattctctcctctcatctttgtacttgctggcaactctggtaaactgttggttaataaggaggaaggtggagacaaacaagctagttgagatgtgaatcaacaggaagcaccacacgtagtggttgagagtgtgaactctaggactagagagactgggcccagtttctgaatctgtcatggaccagttgggtggtcatttgcaagttaattcagttccctgtttcttagttttcctgctagtgaaataggaagaataatagtaaccagagtagttttaggattaagtaaaatgatgttttttaatagtcttggttattgttaggacaagtaataacaacaataacattttactttcctgttgtgctaattgttgttaaatattttatcccatgattgatcctcccaatgattttgttatccccatttgataaaggaaaaatgttgatggtcacgtggtttatgggatatgcccaagggtaggcttggtgagtaagagagctgggatttaagtccagttcaaaccctcatatatggaatataaacaatgataaacaaacaaataaacaaaaacaaagtacatgaacacaccaaactgaacaaaaacacatagacatggagaaaggagcgatggttaccaaaggggaaggggtgaagggggaggcgaaatgggaaaaggggatcaactgcatgggaatggatggagaataaattttgtgtggtgagcatgatacagggtagatagaaatagaactagtatgttgtacacatgaaacatacaacgttgtaagttaacattacctcaataaaataaataaataaattcagtccatccagttccaattctaatgttctttctgtcattcagaaaacgctctgcccaatcttctggtctcctattctttgcccatgagggatcaatctatgtgttccaggtatggagagttttacatagtaatcaggaatttatgtaacccttggaagagccaaaggtcagagaagccaccagagatctcagcctgaaacactaaagccataagtctaggagttcccatcgtggctctgcggaaacgattctgactagcatccatgaggacgtaagttcgatccctggcctccctcagtgggttaaggataaggcattgctggtgagctgttgtgtaggtcgcagatgcgcctcagaatccacattgctgtggccgtggtgtaggccagcagctacacttccgattggacccctagcctggaaacctccatatggccgtgagtgtggaagaagaagaggaagaagaagaggaagaggaaaaagaaggaggaggaggaggaggagaagaagaagaagttgttcttaggaactggtggaaaggtgctgtgaagctccatctatccaggtgtccaaatacaaccacctctgcaggataaaggctctgtttttcttcttcctcccaaatgtttctcatagatgctctaccctggatcctgcaggcaaaggcattctgggaaaaatagtctctgctttgcatctgcaatgcanaggagatgggagtgatgctgaattgatagccaaaaatccagcatacagccgagggattcattcagtcactancaaattataggggtcaggaaatgtcctgtgggaaatgtcctgggtccaaatgcccctttcanaatgtataattatccatgggcaagttactgtaccactcaggctctttgcctatcagagttatagaaaataacactaactgcatgagtctcaccaaacccggaactcagtgaatgctcagtattggttgntgctcccaacagcggtaaactgactcatgtactctgagggctggttgagctgagtgctctttaatagtaccagacaaatcctctttgggcagagaggccagttggccctatatccattattccttcctccatagtgatagaggccatgtgatggcccaggtgaaggttacatttctggcctgctatttattcttattagggcctcacttgtaggctatggacgttcccaggctaggggctgaattggaactgcagctgctggcctacacgwcagccacaataacatgagatctgagccacgtacgcgacctaccctgcagcttggggcaacaccggatccttaacccaatgagtgaggccagggattgaacccacatcctcatggatactagtcaggttcgtaacccactgagccacagtgggaactctgatatttctagtctctttggaatccaagtctggctcccatcttacctgtggctgtgtgtgtgggtgaggatagtgatgcgggcacactccaggtctggcctttgaaaggaactgaatctgcacccccttcttctccctctttctgctggttggatatggccagggcaccgtggctctcaaggactgtgggctaacgtggtgaattgaaggcagcagagcagggctccaggaggaattgagctttagtccctctaggactgctgcgtccaacaagagagcttggactcagactactagatgggtgaagaatatacttctatcttgttggacccctgaagttgctctgctgtagcctatcttctaactatatattactggattcagaggctccaacattattcttcaattatgatataaaggcagctttctgtaggtgtgcgaatgctcacttgagacagatttcaagccagaatggccagtatctcacttagccagctacctatattttgaaagacagatcctgaaagggatgggtgacaaaagaagtgcaaaaatggggagaggtatagcgccaggaagcaattccgtaggcgcttagatgtcctacagggggaagcaggcagtcctttaaagcagccaggtgtgcagctcagaatggcaggaggggtgataagaaatgctttaaatgcacaagtcgaatactggcctttaaagcatttgctggcctttgtgaatactgctggtattcaaacaaacaaacttgacggacaaagcagtttccctcagctgatgcccgcgtgatcatcttcgcttagctgagcaccacgcatgcttctaccaataatcataatttaagaattgcaagtgataaaagcatcacactgaaatatctgtagcctttacagacatgacgaactggaaagtactgccacagtcgttgcgatcttggctggcacgtttattgtggctttaactacatcgtaacctcatcagttgtatagctgtcctttctctagactgtgaaccctctgtgaattatttgcctttgtaccttccactccctggatggtcgataaatatttaaatggataaatctgttcaacctcttcacttcatagtgacttgttcaaagtcgaaattaataatagaaaaaaaaatctgctggcttttttccttctaagtagagagttctagttctcttttccccatggcttggagggttaagacaaatttcttattaggggtgccataacccagatccacatacacacacaagcccccaaaacgcatacacacattcacaaaaacacacaggtacgctaacaaacggcaaacaaccataagcacagaggcctacacacaaagaaaaaccacaaacccatcacatggtttcacacttgaattttgggtcaacaacaagtacttattagtataaataagtcttaaatattgcacacacaatacacatgggacatatgtatgctaaaaggattgtttgctgtttatcctggaattccaatttagctgtagaatctgtacttattttatttttatttttatttatttgttttgctttttcaggcccgcacccagagcatatggaggttcctaggctagggtcgaattggagctacagatgccggcctataccagccacagcaatgcaggatccaagttgcatctgtgacctataccacagctcacggcaatgccagatccttaacccactgagtgagaccaaggatcgaacccacaaactcatggttactagtcagattcgtttccgctatgccacaatgggaactctgggacctgtaattttatctgctaaatctgctaaccttaactcagacacacacatagatacacacatagatacatacagataaatgtaagcaagtacacacacaaacacgtacactcccacctatttagttgttcccatcagagctggccactaccaagatggtcattagaatcccagttctaggagttcctgtcgtggctcagtggttaacgaatccgactaggaaccatgaggttgcaggttagatccctggcctttctcagtgggttaaggacccggcgttgttgtgagctgtggtgtaagttgcagacgcggcttggatcccgcgttgctatggctctggtgtaggccggcggctgcagctccgattcgacccctgatcttggaacctacatgtgccaagaaatggcaaaaaagaaaaaaaaaaaaaacaaaaaagaaaaaagaaagaatctcagttctagttttccctccgagagctggaggtgtcaaggaggtttgggaaggaagcagaggttgattctctgctcatagttgtctgcaggaggagaaaaaaggagtcttatccttcagcgatagaagcagctgctttagaaagggcacacctctcccaaagacttgcacacacacacacacacacacacacacacacacacacacacacagcctctgtaacacccttaacaattccttctgcatgattttgtaaagaggttcaagctccagagaggagcttttttccttagaaatctgtgatgctgagtttacagatatgcttggcaatgaatgatggggtgggtttgcatgaaattctacctctgttgcgggtgctaaaatgtacccctaacttagcccatgggtaattcttggctcttcttactttattgccctccccagcccataaccattttccattacatacccacgagggtctactgacttcgcctcagaaatgcttggagaaggttttgccctccactcccagggccactggccatcctttgccccttaccacctctcacctgaagatgacctcagccctattgcctcaccccagcccaccgactaattcatcagaatgaggcacctgatgtgcagatttgatctcgttcctcccttcctttgatgggcactctatttgcttcaccataaacccagacatctcggcatggcctccaaggcttttcaccatcagaagccattccaacttctgtttccaccttctgcctcagacattcaggctggcctcacccaaaggaattgtttccccacaaacactcagccccttctcttgctcagctttccccccattattcttcacctcctttccagcctagtgaattaattacccttccgaccagtttacacaccctttccaaccaaccccacactgcctccccctgcctgtagaattaatcactcccttttcaatgaacccacagaattttgcatatacatgcttctttctctcttaccatggtatcaagtaagatgtgcagaccactctctctcacaaacctgtgagcccttctggggcagaggttaacgcttattcgtatacaaaccaccagtatccaggatagtgcctgctacagaagaggttccgacaaaatgatgttggtagccatgtgcattggggttaagaggggctttggagtcaaagagaccaagtttggtacctagaaaataccattcgttagctccaggacttgcagtaggttatgcagtatctctgtggtaggtagagtactagctccttccccccaaacatgttcgtgtcttactccttggaccctagggacctgtaagagaaattcagattgcagatgaaattaaggttgctaatcagttgaacttaaaatagggagaatagttcagattacccaggtgggcccaatacaatcacaaaggtccttaaaagtgaaaggctgaggcagaaaaggggtccatgtgatgcaacatgaggaatgactcaacccaactttgctggttttgaagctggagggaggctaatgcaaggactgtgggcagcttctagaaaactaggaaagacatggaaatagagtctcccctaaagcctccggaaaaacatgcagcactgcaaacgcctaggtgcaaatcaaaaattatatgttacatgacttgagaaggcttaaaaagtagaaaagttctgattctttctctggagcactaggcctcaatgagttacttttccatgaagaatgtctagaaaaactcaataattctcagtttaccctcctccaagagagatatatatatatcttttgtttttagtaaaagagagtccgtctctgaaatcctgatataacatattaaatatatatatatttaatatatatatatcttttgtttttagtaaatgagagtccatttatatctatatacttatatctctatatatcttttgtttttagtaaaagagagtccatttctgaaatcctgatagaacatattaaatatatgtttttaatgaataaatatttttaatgaaaatgtatatttagtgaacagatatatcaccaacaatgccaccgcccagagtaatcatacttgacgttttggagactataattttaggggtgtgcatgtgtatgtgtttgtgtgtgcgtgtgtatctgggcatctacaaacatggcataacatcttaaaatctttacaagttttccttatcaggcacataacacaaacagccttgccatttatttttcttctttgacatccctcagaaaaaatttggagtctttagaaaggtctaacacatttctaattatatttagtcttcaaaattttatgtttgtgtagctactgtgaagcttttctattgcatgttctaagaggttattgaggatgtggctatattttttcattatgtatactaaaattatctttctgtactctcttagttctaatattttttttcaataaattcccagatattttccaaagaagcaattatgttttctaaaaataatggtaggagttcccattgtggcacagtggaaacgaatccaactaggaaccatgaggttgcaggttcgatctctggcctccctcagtgggttaaggatctggcattgctgccagctgtggtgtaggtcgtagatgtggctcagatcctgtgttgctgtggctgtggtgtangccggcagctgtagctctgaatagacccctagcccgggaatctccatatgccgtgggtgcagccctaaaaaagacaaangacaataaattaaataaataaataaataaataaataaaataatagtaaatctgctccaatagtcatagctctcatttaattttctatttaacttagattctgtgacctatttcaagagaaaaaaaaggagtctccccacggacatctcctggaggactgggaaatccttccaaaatattccagaatcagctttttggtcttttgcaaatttaacttctttaaacttcgcagaaacattttattcatttatttatttatttacttccatttgcttttagggccatacctgcagcaaacggaatttctctggctaagggccaaatcatagttacagttgccggcctacaccacagccacagcaacgccagatctgagggacacaactgctctaaagctgctgttaacccagacatttcctcaggagggtgtggtgacttcaggaccctcagacgttggttcccgagtggcccctggaaaagggactgtcctttctgtttgtccccaggcagccccccccacctggacgctgctggggggacagcaggcccgacacactggagctgaaggcaggctccttggggggggggtgagatgaggagctgcctcccccgtggtccccgcctctgctccatccgggcatttacgtccacggggagctcccgtgtcctttccccagaccagggcagggggtgttctgagggcagggactgtgagggacatctgggaagatcaggaaagggagtctggatggaggagaacggggtggcattattgattttattgtagcaataatcagggggctcaagggccgaagtggaattgaccttgagaaggaggacacgtcccaagcggcctcccctgcggtcatcaggcaggaggcctgagggagcaggtggctgagggcggcctccctggttctgcaggaggtgcgaggctggtctaccctggggacctgagggcccctgtaagagagaagtgggagggagaggacaggaagtcctcccctgtcccttagagaaaggtgagtgatggacgtgtaatagaaaattggattcccaggagtcctagcagttaaggatttggtattgtcactgctatggcttgggatcgatccctggcccaggaacttgtgcatgctacaggcacagccaaaaaaaaaagtaatagagagctggccaaaaatatggctaaatcagtatttcacaaaaaagcagcagttgaactagatgcataagaaaggagatgaagtccactggcaacaggagaaatacgtattttaaaaatatatattattttgaagctttctattcagcaaggcatttggaaagactggtaaaattcagagtcgtttaatgtcgcacaatgggctctctcgcctcctggctggcgtgtaggtggtgacggtccttggaggccgttggtggccgtggcttttcatggatgaatgtcttccatcctagagacagaccgtgatttatttatcccttcagcagaggatggacctttgggtgcttctcctctctttccttttggagtaattctgctctgaatattcacggaaatttcccgtgtggacctaagtcttcatttccttggggaaacacctggtggaaagctgggccacatggaaaatcctgagaaaggggctgagaaccggctcaactgtcttgcactggcaggtctgctgggttcttgtaactggggagctctcagggacagagccatgtggtctcagtataattagggaagaagtggcgacacctgcacacgtgtcatccttgtggacacgcccacacacaggggtgtgcactgcagctgccccgccagtgccgacgcacacctggagcagactgggggctctttgtgtggctttgccctgagggacatgggacagctcttagagagaccgggcagctgggagctcagacctcgtcctgattcagccactgttaccctgtgacgctgggcaagtgccttggtgtctgtgggtcttcttagttcctgtcctgtccaaggtgttgtgaggctcatgtgggttcatggagctgaaagtgcagtagcttaaccccaggcagggcggctccccaacaggaggtcctggtgcttgtggccaatctgttacccttcatccaggatgacagaaaggggacaagaagagaagaaagcaggcgtcgtatcccagtctaggtggggaaagacccagtgagaaaggccagggtgctgagcatgcacccctccctctgagcaagccaaggcccccagcacctcagcccctgcagccctgctggcctgggccccagggggagggaggagatcaggttgcagcggctgggctatatagccccagcctccagggccaggctc

SEQ ID NO: 4 - DNA, 962 bp, Mammal
  promoter (1)...(962): Consensus sequence
ggtccttggaggccgttggtggccgtggcttttcatggatgaatgtcttccatcctagagacagaccgtgatttatttatcccttcagcagaggatggacctttgggtgcttctcctctctttccttttggagtaattctgctctgaatattcacggaaatttcccgtgtggacctaagtcttcatttccttggggaaacacctggtggaaagctgggccacatggaaaatcctgagaaaggggctgagaaccggctcaactgtcttgcactggcaggtctgctgggttcttgtaactggggagctctcagggacagagccatgtggtctcagtataattagggaagaagtggcgacacctgcacacgtgtcatccttgtggacacgcccacacacaggggtgtgcactgcagctgccccgccagtgccgacgcacacctggagcagactgggggctctttgtgtggctttgccctgagggacatgggacagctcttagagagaccgggcagctgggagctcagacctcgtcctgattcagccactgttaccctgtgacgctgggcaagtgccttggtgtctgtgggtcttcttagttcctgtcctgtccaaggtgttgtgaggctcatgtgggttcatggagctgaaagtgcagtagcttaaccccaggcagggcggctccccaacaggaggtcctggtgcttgtggccaatctgttacccttcatccaggatgacagaaaggggacaagaagagaagaaagcaggcgtcgtatcccagtctaggtggggaaagacccagtgagaaaggccagggtgctgagcatgcacccctccctctgagcaagccaaggcccccagcacctcagcccctgcagccctgctggcctgggccccagggggagggaggagatcaggttgcagcggctgggctatatagccccagcctccagggccaggctc

(19) United States
(12) Patent Application Publication
(10) Pub. No.: US 2005/0043530 A1
(43) Pub. Date: Feb. 24, 2005

(54) Seminal vesicle tissue-specific promoters and uses thereof

(76) Inventors: Marc-Andre Sirard, Quebec (CA); Francois Pothier, Quebec (CA); Michael Dyck, Quebec (CA); Dan Lacroix, Quebec (CA)

Correspondence Address: EDWARDS & ANGELL, LLP, P.O. BOX 55874, BOSTON, MA, 02205, US

(21) Appl. No.: 10/499,406

(22) PCT Filed: Dec. 19, 2002
(86) PCT No.: PCT/CA02/01969

Related U.S. Application Data
(60) Provisional application No. 60/341,287, filed on Dec. 20, 2001.

(51) Int. Cl. C07H 21/04

(52) U.S. Cl. 536/24.1

(57) ABSTRACT

The present invention relates to genetically modified animals for the production of recombinant proteins. Particularly, pig's seminal vesicle and sexual accessory glands are genetically transformed to recombinantly produce such protein of interest in the semen of sexually mature pigs. Even complex proteins can be produced in large quantities and purified. The present invention also relates to mammal seminal gland tissue-specific promoters that can be used to make gene constructs including a protein-coding sequence not natively associated with the promoters or a sufficient portion of the promoter such that the portion actuates the preferential expression of the protein-coding sequence in the seminal vesicle and/or accessory tissues of mammals.

### TECHNICAL FIELD

The invention relates to the production of recombinant proteins in pig semen. Particularly, this invention relates to transgenes that comprise at least a semen-specific protein promoter operatively linked to a DNA sequence coding for a signal peptide and a desired recombinant protein product. When such a system is transgenically incorporated into a pig, the recombinant protein is expressed in the semen of the animal. This invention also relates to the transgenic animals that produces the desired recombinant product in its semen.

### BACKGROUND OF THE INVENTION

The eukaryotic cell culture expression system has several advantages over the bacterial, yeast or Baculovirus expression systems. Bacteria do not, and yeasts only in a limited manner, carry out post-translational modifications of an expressed proteins. Eukaryotic cells are able to perform sophisticated protein modifications, which are often necessary for the proper function of proteins.

Although insect cells are potent transformation vehicles for higher eukaryotic proteins and generally perform modifications of proteins at a certain level, the cost for culturing those cells is much higher than that for eukaryotic cells. In addition, the host cells are eventually lysed by insect cells and thousands of host proteins along with the expressed transformation protein are mixed and released into the culture medium, which makes purification of the expressed transformation protein difficult.

A number of laboratories have reported tissue-specific expression of DNA encoding various proteins in the mammary gland or the production of various proteins in the milk of transgenic mice and sheep. For example, Simmons, J. P., et al. (1987) Nature 328:530-532 report the microinjection of a 16.2 kb genomic fragment encoding beta-lactoglobulin (BLG) including 4 kb of 5′ sequence, 4.9 kb of the BLG transcription unit and 7.3 kb of 3′ flanking sequence into fertilized mouse eggs. According to these authors, the sheep BLG was expressed in mammary tissue and produced BLG in the milk of the transgenic mice at concentrations ranging from about 3.0 to about 23 mg/ml.

Expression in a tissue specific manner has been accomplished in several species of farm animals, including bovine, ovine, caprine, mice, and porcine, mostly through production of recombinant proteins in milk and blood. The U.S. Pat. No. 6,201,167 described the use of semen vesicle specific promoter to regulate the expression of recombinant human growth hormone (hGH) in the semen of transgenic mice. Although this approach defines the possibility of producing recombinant proteins in the semen of transgenic mice, limited number of promoters of murine origin have been tested.

Promoters control the spatial and temporal expression of genes by modulating their level of transcription. Early approaches to different genetically engineered organisms, either plants or animals, utilized strong constitutive promoters to drive the expression of foreign genes.

As strategies in eukaryotic biotechnology have become more sophisticated, there are requirements for specific promoters to target transgene expression to a particular tissue at a particular developmental stage.

Recombinant DNA technology has enabled the cloning and expression of genes encoding medically and agriculturally important proteins and glycoproteins. Such products include, for example, insulin, growth hormone, growth hormone releasing factor, somatostatin, tissue plasminogen activator, tumor necrosis factor, lipocortin, coagulation factors VIII and IX, erythropoietin, the interferons, colony stimulating factor, the interleukins, urokinase, and antibodies.

Many of these important proteins, however, are large (molecular weights in excess of 30 Kd), secreted, require sulfhydryl bonds to maintain proper folding, glycosylated and sensitive to proteases. As a result, the recombinant production of such products in prokaryotic cells has proven to be less than satisfactory because the desired recombinant proteins are incorrectly processed, lack proper glycosylation or are improperly formed. Accordingly, production of these recombinant proteins in cultured eukaryotic cells has been resorted to. This technique has proven to be both expensive and often unreliable due the variability of cell culture methods. For example, average yields are 10 mg of recombinant protein per liter of culture media, with the resulting cost typically exceeding one thousand dollars per gram of recombinant protein.

Harvesting from body fluids as opposed to solid tissue is desirable, because such routes, are by and large renewable, and most proteins of biomedical importance are themselves secreted into body fluids. Secretion into the bloodstream is a possible route, either from liver or B lymphocytes, but the coagulating properties of blood and the presence of biologically active peptides and antigenic molecules may prove a hindrance to subsequent downstream processing.

U.S. Pat. No. 6,201,167 discloses transgenic mice producing human growth hormone (hGH) in the semen under control of the promoter p12. While this demonstrated the production of recombinant proteins in mouse semen, the mouse remains a laboratory model only. Transposition of such an application between mouse models and a farm animal, mostly those producing a very high volume of semen, cannot be done without extensive experimentation. It is known in the art that transgenic animals have within their cells mechanisms that can influence, or even prevent, the expression of a transgene, such as DNA methylation or deletion from the genome (Kappell et al., (1992) Current Opinion in Biotechnology 3:549). Furthermore, position effect and unidentified control elements also are recognized to cause aberrant expression (Wall et al., 1996, Theriogenelogy, 45:61).

U.S. Pat. No. 6,201,167 does not clearly ascertain that subunits of dimeric proteins or peptides can be produced simultaneously in the semen of an animal far larger than the mouse and having different physiological mechanisms at the level of the seminal vesicle and sexual accessory glands.

Considering the above-described state of the art, these is still place for the development of new regulatory nucleic acid sequence or promoters controlling the expression of recombinant protein of interest in a tissue specific way, as it is still highly desirable to be provided with a new means to produce recombinant proteins in large quantities.

### SUMMARY OF THE INVENTION

One object of the present invention is to provide an isolated DNA sequence which regulates the expression of a heterologous gene in a mammal seminal vesicle and accessory tissues thereof, wherein the isolated DNA sequence is selected from the group consisting of SEQ ID NO:1, SEQ ID NO:2, SEQ ID NO:3, SEQ ID NO:4, and a functional fragment thereof, or a DNA sequence having at least 80 percent of homology with the isolated DNA sequence.

The mammal may be a human, a porcine, a bovine, a primate, a caprine, an ovine, an equine, a murine, a canine, or a feline.

In accordance with the present invention there is provided a DNA construct comprising a DNA sequence encoding a heterologous RNA or protein in operable association with an isolated promoter having an isolated DNA sequence as defined above, or a DNA sequence having at least 80 percent of homology with the isolated DNA sequence. The RNA may be an anti-mRNA or a ribosyme.

Another object of the present invention is to provide a mammalian cell having a genome which comprises a DNA construct as defined in claim 3. The mammalian cell can be a seminal vesicle cell or a cell from accessory tissues thereof, and the cell may come from a human, a porcine, a bovine, a primate, a caprine, an ovine, an equine, a murine, a canine or a feline cell.

Again, another object of the present invention is to provide non-human transgenic or chimeric mammal whose seminal vesicle or accessory tissues thereof contain a DNA sequence encoding a heterologous protein in operable association with a DNA construct as defined above, a fragment thereof or a DNA sequence having at least 80 percent of homology with the isolated DNA sequence, wherein the transgenic or chimeric mammal is naturally or artificially physiologically induced to express the DNA construct in seminal vesicle cells or accessory tissues thereof such that the heterologous protein is detectable in semen or seminal secretion produced by the transgenic or chimeric mammal. The expression of the DNA construct into the non-human transgenic or chimeric mammal may be artificially induced by hormone administration.

Also, another object of the present invention is to provide mammal whose seminal vesicle cells or accessory tissue cells thereof have a genome comprising a DNA sequence encoding a heterologous protein in operable association with an isolated DNA sequence as defined in SEQ ID NO:1, SEQ ID NO:2, SEQ ID NO:3, SEQ ID NO:4, and a functional fragment thereof, or a DNA sequence having at least 80 percent of homology with the isolated DNA sequence, wherein seminal vesicle cells or accessory tissue cells thereof are naturally or artificially physiologically induced to express said DNA construct such that the heterologous protein is detectable in semen or seminal secretion produced by the mammal.

In accordance with the present invention there is provided a method of producing a recombinant protein comprising the step of:
introducing into a cell a DNA construct as defined in SEQ ID NO:1, SEQ ID NO:2, SEQ ID NO:3, SEQ ID NO:4, and a functional fragment thereof, or a DNA sequence having at least 80 percent of homology with the isolated DNA sequence, to form a genetically transformed cell;
naturally or artificially inducing said genetically transformed cell of step a) to express said DNA construct such that the heterologous protein is produced.

The genetically transformed cell can be further cultured in vitro or naturally occurring in a living organism.

The heterologous protein may be produced in a culture medium in which said genetically transformed cell is cultured or in the semen of a mammal containing the genetically transformed cell.

Another object of the present invention is to provide the use of an isolated DNA sequence as defined in SEQ ID NO:1, SEQ ID NO:2, SEQ ID NO:3, SEQ ID NO:4, and a functional fragment thereof, or a DNA sequence having at least 80 percent of homology with the isolated DNA sequence, for regulating the expression of an heterologous RNA or protein in a human or an animal.

The present invention provides a new method for producing large quantities of recombinant protein products in the semen of genetically modified animals.

According to one embodiment of the present invention, a DNA sequence coding for a desired protein is operatively linked, in an expression system, to a genital tract-specific (protein) promoter, or any promoter sequence specifically activated in male genital tissue, through a DNA sequence coding for a signal peptide that permits secretion and maturation of the desired protein in the genital tract tissue. Preferentially, the expression system also includes a 3′ untranslated region downstream of the DNA sequence coding for the desired recombinant protein. This untranslated region may stabilize a rDNA transcript of the expression system. Optionally, the expression system also includes a 5′ untranslated region upstream of the DNA sequence coding for the signal peptide.

The transgene, or expression system, is transgenically introduced into a pig host genome. As a result, one or more copies of the construct or system become incorporated into the genome of the transgenic animal. The presence of the expression system permits the male pigs to produce and to secrete the recombinant protein product, into or along with its semen. Such method permits the production of the desired proteins.

For the purpose of the present invention the following terms are defined below.

The expression “genetically modified”, as used herein, is intended to transgenic or chimeric animals. Transgenic animals means animals having a transgene in all their cells, and chimeric means animals having a transgene in some cells, groups of cells, or organs.

The term “gene”, as used herein, is intended to mean a DNA sequence responsible for the production of a protein (or polypeptide). This includes first and foremost the actual coding sequence, which dictates the specific order of amino acids in a polypeptide. In eukaryotes (nucleated cells, such as those of mammals), that sequence may be distributed in short stretches of DNA called exons, interspersed with non-coding DNA sequences called introns. Those sequences, exons and introns, may be transcribed into a pre-messenger RNA, from which the introns may eventually be removed before the mature MRNA is translated into the actual protein encoded by the gene.

The term “promoter”, as used herein, is intended to mean non-coding regulatory sequences for transcription, usually located near the start of the coding sequence, which may be referred to as the gene promoter or the regulatory sequence. Put into a simplistic yet basically accurate way, it is the interplay of the promoter with various specialised proteins called transcription factors that determines whether or not a given coding sequence may be transcribed and eventually translated into the actual protein encoded by the gene in a given tissue.

The term “vector”, as used herein, refers to a nucleic acid, e.g., DNA derived from a plasmid, cosmid, virus, artificial chromosome or bacteriophage or synthesized by chemical or enzymatic means into which one or more fragments of nucleic acid may be inserted or cloned which encode for particular genes. The vector can contain one or more unique restriction sites for this purpose, and may be capable of autonomous replication in a defined host or organism such that the cloned sequence is reproduced. The vector may have a linear, circular, or supercoiled configuration and may be complexed with other vectors or other material for certain purposes. The components of a vector can contain but is not limited to a DNA molecule incorporating DNA; a sequence encoding an excision protein or another desired product; and regulatory elements for transcription, translation, RNA stability and replication.

The term “heterologous protein”, as used herein, is intended to mean any protein, peptide, polypeptide, that is not natively associated with the promoters of the present invention. However, a heterologous gene encoding for the heterologous protein may therefore originate from an animal or a human in which it is recombinantly expressed.

The expression “functional fragment”, as used herein, is intended to mean a nucleic acid fragment having at least enough molecular elements to a heterologous coding sequence to confer a tissue specific expression targeted to seminal glands and accessory tissues thereof. For example, but not limited to, a fragment may have a length of 1000 kb starting from the 3′ end of a non-coding isolated DNA sequence of the present invention for regulating the expression of a heterologous coding sequence in cells of the seminal gland. The same 1000 kb fragment can be linked to any other regulatory elements, such as transcription regulatory elements, from different origins coming from or other species, in the preparation of a seminal gland tissue specific promoter. Anyone skilled in the art should be capable or recognizing such other regulatory elements.

The expression “operatively linked”, as used herein, is intended to mean the linking of a genital tract-specific promoter or a promoter specifically activated in genital tract tissue to a DNA sequence coding for a desired protein, to permit and control expression of that DNA sequence and production of that protein in the accessory glands of a male genital tract.

The expression “recombinant protein”, as used herein, is intended to mean a protein or peptide encoded by a DNA sequence that is not endogenous to the native genome of the animal in whose semen it is produced in accordance with this invention. The term also applies to a protein or peptide encoded by a DNA sequence which is endogenous to the native genome of the animal in whose semen it is produced does not lead to the production of that protein or peptide in its semen at the same level that the transgenic animal of this invention produces that protein in its semen.

The expression “genital tract”, as used herein, is intended to mean the reproductive anatomical male system whole or in part involving the prostate gland, seminal vesicle, epididymis, seminiferous tubules, ampule, vas deferens, and bulbourethral gland.

### BRIEF DESCRIPTION OF THE DRAWINGS

FIG. 1 illustrates pFSH RIA results in mouse seminal fluid after in vivo transfection;

FIG. 2 illustrates an effect of age on pFSH production level in mouse plugs;

FIG. 3 illustrates an effect of soak time on detected pFSH levels in mouse plugs;

FIG. 4 illustrates the strategy for preparing the transgene pGP8-FSHα;

FIG. 5 illustrates the strategy for preparing the transgene pJ3P8-FSHβΔ;

FIG. 6 illustrates the restriction map of the transgene pGP8-FSHα; and

FIG. 7 illustrates the restriction map of the transgene pJ3P8-FSHβΔ.

### DETAILED DESCRIPTION OF THE PREFERRED EMBODIMENT

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

In accordance with the present invention, there is provided new promoters allowing the expression of desired proteins or polypeptides specifically in the semen of any mammal through the regulation of the transcription at the level of the seminal vesicle, sexual glands and/or accessory tissues thereof.

Also, with the present invention, new promoters elements useful in creating transgenic mainmals are described. These promoters, identified here as the PSP1, PSP2 and AQN-3, are from pig and can regulate the expression of a contiguous gene or coding sequence only in the seminal vesicle and accessory tissues thereof of any mammal. The sequence of PSP1 is set forth in SEQ ID NO:1, PSP2 in SEQ ID NO: 2, AQN-3 in SEQ ID NO:3, and concensus sequence in SEQ ID NO:4. These sequences reside respectively on 8,980, 13,126, and 15,572 base pair DNA fragment and contains all sequences sufficient to confer tissue-specific expression, when compared to a protein-coding DNA sequence, and probably some additional unnecessary DNA sequence. Mammal seminal vesicle and accessory tissues promoters are thus available, i.e. containing either SEQ ID NO:1, SEQ ID NO:2, SEQ ID NO:3, SEQ ID NO:4, or a portion or version of SEQ ID NO:1, SEQ ID NO:2, or SEQ ID NO:3, SEQ ID NO:4 sufficient to effect preferential expression of a transgene in the seminal vesicle or accessory tissues in a mammal.

One use of a seminal vesicle tissue-specific promoter, such as those of the present invention, is to control the expression of target genes in transgenic non-human mammals in order to attempt to produce in the semen recombinant proteins, peptides or polypeptides of interest, from which it can be extracted or have any effect on the quality of the semen itself. For example, one of these promoters can be used to direct the expression of a gene encoding a pharmaceutical protein or peptide factor that is therefore extracted, purified and used in a prophylactic or therapeutic application. All or part of the base pairs of SEQ ID NO:1, SEQ ID NO:2, SEQ ID NO:3, or SEQ ID NO:4 may be used to direct the expression of such foreign or endogenous genes in either the sense or antisense orientation, for over-expression or suppression of native gene expression. The genes driven by the PSP1, PSP2 or AQN-3 promoter may be of different origin, as for example, but not limited to, an animal, plant, or microbial protein.

The seminal vesicle and/or accessory tissue promoters of the present invention may also be used to control the expression of target genes which affect the quality of the semen without affecting the physiology of the reproductive glands or accessory tissues. For example, but not limited to, an anti-oxidant peptidic factor can be recombinantly secreted by transgenic seminal vesicle cells to preserve the degradation of a specific product, such as sperm membrane, or a product of the seminal fluid that could impair the quality of the semen after degradation.

In particular, in an expression vector or a transgenic animal according to the present invention, the sequence encoding a protein of interest can be proceeded toward its 5′ end, by a sequence corresponding to the promoters of the complete pig PSP1, PSP2 or AQN-3 promoter, or by an equivalent sequence, which ensures the function of the promoter. “Equivalent sequence” is understood to preferably designate a sequence having at least a length of 2 Kb upstream from the 3′ end of the PSP1, PSP2 or AQN-3 promoter and in particular comprising the expression elements situated on the fragment with a length of at least 1 Kb upstream from the 3′ end of the complete PSP1, PSP2 or AQN-3 promoter. Among the genital tract-specific protein promoters useful in the various embodiments of this invention are the p12, p25, kallikreins, PSA, SBP-C, GP8, J3P8, PSP-1, PSP-2, AQN-3, and secretory protein IV promoters. The genital tract specific protein promoter or the promoters that are specifically activated in genital tract tissue may be derived from either cDNA or genomic sequences. Preferably, they are genomic in origin.

Any of the cell types defined herein can be altered to harbor modified nuclear DNA of the present invention. A type of cell that can be considered in the invention may be, for example, an embryonic cell, a stem cell, a founder cell, a zygote, or a gamete. The cell targeted for genetic transfonnation may be manipulated in in vitro as well as in in vivo conditions, through direct transfection in a petri dish, or by injection of DNA constructs into a living organism.

Examples of methods for modifying a target DNA genome by insertion, deletion, and/or mutation are retroviral insertion, artificial chromosome techniques, gene insertion, random insertion with tissue specific promoters, homologous recombination, gene targeting, transposable elements, and/or any other method for introducing foreign DNA. Other modification techniques well known to a person of ordinary skill in the art include deleting DNA sequences, and/or altering nuclear DNA sequences.

When expression of the DNA of the transgene is necessary to generate a desired phenotype, e.g. to produce a recombinant polypeptide, the transgene typically includes at least a 5′ and preferably additional 3′ “expression regulation sequences” each operably linked to a recombinant or secretory-recombinant DNA as defined hereinafter. Such expression regulation sequences in addition to controlling transcription can also contribute to RNA stability and processing, at least to the extent they are also transcribed.

Such expression regulation sequences are chosen to produce tissue-specific or cell type-specific expression of the recombinant or secretory-recombinant DNA. Once a tissue or cell type is chosen for expression, 5′ and optional 3′ expression regulation sequences are chosen. Generally, such expression regulation sequences are derived from genes that are expressed primarily in the tissue or cell type chosen. Preferably, the genes from which these expression regulation sequences are obtained are expressed substantially only in the tissue or cell type chosen, although secondary expression in other tissue and/or cell types is acceptable if expression of the recombinant DNA in the transgene in such tissue or cell type is not detrimental to the transgenic animal. Particularly preferred expression regulation sequences are those endogenous to the species of animal to be manipulated. However, expression regulation sequences from other species such as those from bovine, caprine, ovine, murine, rabbit, avian or even human genes may also be used. In some instances, the expression regulation sequences and the recombinant DNA sequences (either genomic or cDNA) are from the same species, e.g., each from porcine species or from any other mammalian source. In such cases, the expression regulation sequence and the recombinant DNA sequence are homologous to each other. Alternatively, the expression regulation sequences and recombinant DNA sequences (either cDNA or genomic) are obtained from different species, e.g., an expression regulation sequence from porcine species and a recombinant DNA sequence from a human source). In such cases, the expression regulation and recombinant DNA sequence are heterologous to each other. The following defines expression regulation sequences from endogenous genes. Such definitions are also applicable to expression regulation sequences from non-endogenous, heterologous genes.

Other transcription regulation elements can be incorporated into the vectors or constructs of the present invention. Other 5′ and 3′ regulatory elements may be incorporated according to different arrangements into the expression vectors of the invention. In general, a 5′ expression regulation sequence includes the transcribed portion of an endogenous gene upstream from the translation initiation sequence (the 5′ untranslated region or 5′ UTR) and those flanking sequence upstream therefrom which comprise a functional promoter. Such sequences typically comprise a TATA sequence or box located generally about 25 to 30 nucleotides from the transcription initiation site. The TATA box is also sometimes referred to the proximal signal. In many instances, the promoter further comprises one or more distal signals located upstream from the proximal signal (TATA box) which are necessary to initiate the transcription. Such promoter sequences are generally contained within the first 100 to 200 nucleotides located upstream from the transcription initiation site but may be farther from the transcription initiation site. Such sequences are either readily apparent to those skilled in the art or readily identifiable by standard methods.

In addition to such 5′ expression regulation sequences, it is considered that additional 5′ flanking sequences (referred to herein as “distal 5′ expression regulation sequences”) also be included in the transgene. Such distal 5′ expression regulation sequences are believed to contain one or more enhancer and/or other sequences which facilitate expression of the endogenous gene and as a consequence facilitate the expression of the recombinant or secretory-recombinant DNA sequence operably linked to the distal and proximal 5′ expression regulation sequences. The amount of distal 5′ expression regulation sequence depends upon the endogenous gene from which the expression regulation sequences are derived. The determination of the optimal amount of distal 5′ expression regulation sequence used from any particular endogenous gene is readily determined by varying the amount of distal 5′ expression regulation sequence to obtain maximal expression. In general, the distal 5′ expression regulation sequence can not be so large as to extend into an adjacent gene and will not include DNA sequences, which adversely effect the level of transgene expression.

In addition, a 3′ expression regulation sequences may also be included in a construct or expression vector to supplement tissue or cell-type. specific expression. Such 3′ expression regulation sequences can include 3′ proximal and 3′ distal expression regulation sequences from a desired gene. The 3′ proximal expression regulation sequences include transcribed but untranslated DNA positioned downstream from the translation stop signal in the recombinant DNA sequence (also referred to as the 3′ untranslated region or 3′ UTR). Such sequences generally terminate at a polyadenylation sequence (either from the endogenous gene or from other sources such as SV40) and sequences that may affect RNA stability. Generally, 3′ UTR's comprise about 100 to 500 nucleotides downstream from the translation stop signal in the gene from which the 3′ regulation sequence is derived. Distal 3′ expression regulation sequences include flanking DNA sequences downstream from the proximal 3′ expression regulation sequence. Some of these distal sequences are transcribed, but do not form part of the mRNA while other sequences in this distal 3′ expression regulation sequence are not transcribed at all. Such distal 3′ expression regulation sequences are believed to contain enhancer and/or other sequences that enhance expression. Such sequences are believed to be necessary for efficient polydenylation and contain transcription termination sequences.

One embodiment of the present invention is to provide a DNA sequence that allows the secretion of a recombinant polypeptide, a “secretory DNA sequence” encoding a functional secretion signal peptide that is also operably linked within the transgene to direct secretion of the recombinant polypeptide from one or more cell types within a transgenic animal. In general, a secretory DNA sequence may be defined functionally as any DNA sequence which when operably linked to a recombinant DNA sequence encodes a signal peptide which is capable of causing the secretion of the recombinant polypeptide. Secretory DNA sequences in general are derived from genes encoding secreted proteins of the same species of the transgenic animal. Such secretory DNA sequences are preferably derived from genes encoding polypeptides secreted from the cell type targeted for tissue-specific expression, e.g. secreted semen proteins for expression in and secretion from seminal vesicle or accessory secretory cells. Secretory DNA sequences, however, are not limited to such sequences. Secretory DNA sequences from proteins secreted from other cell types within the species of transgenic animal may also be used, e.g., the native signal sequence of a homologous gene encoding a protein secreted other than in the seminal vesicle or accessory tissues.

In another embodiment of the present invention, there is provided a secretory DNA sequence encoding a secretory signal sequence functional in the seminal vesicle secretory cells of porcine species may be used to cause secretion of recombinant polypeptide from porcine seminal vesicle and/or accessory secretory cells. The secretory DNA sequence is operably linked to the recombinant DNA sequence. Examples of such secretory DNA sequences include DNA sequences encoding signal secretion sequences for porcine PSP1, PSP2, AQN-3, AQN-1, AWN, or DQH. The size of the signal peptide is not critical for this invention. All that is required is that the peptide be of a sufficient size to effect secretion and maturation of the desired recombinant protein in the pig genital tract tissue where it is expressed.

“In operably linked” in the context of linking a secretory DNA sequence to a recombinant DNA sequence means that the secretory DNA sequence (comprising codons encoding the secretory signal peptide sequence) is covalently coupled to the recombinant DNA sequence so that the resultant secretory-recombinant DNA sequence encodes 5′ to 3′ for the secretory signal sequence and recombinant polypeptide. Accordingly, the reading frame for the secretory sequence and the recombinant DNA sequence must be covalently combined such that an open reading frame exists from the 5′ end of the mRNA sequence formed after transcription and processing of the primary RNA transcript. This open reading frame in the RNA contains a 5′ sequence portion encoding the secretory signal peptide and a 3′ sequence portion encoding the recombinant polypeptide. When so constructed, the recombinant polypeptide produced upon expression of the secretory-recombinant DNA sequence is of a form that is capable of being secreted from targeted cells, which express the recombinant DNA sequence under control of a promoter of the present invention as described therein.

In other embodiments of the invention, a secretory-recombinant DNA sequence is expressed predominantly in the seminal vesicle cells of transgenic porcine species. Such tissue-specific expression is obtained by operably linking seminal vesicle specific promoters, or expression regulation DNA sequences, to the above secretory-recombinant DNA sequence. Such seminal vesicle specific promoters include the aforementioned regulation sequences from genes expressed in the seminal vesicle or accessory tissue cells of the species. Such seminal vesicle or accessory tissue specific genes include PSP1, PSP2 and AQN-3.

According to another embodiment of the present invention, there is provided a method for gene therapy of seminal vesicle of accessory tissues in a human. In vivo or ex vivo integration of a vector or a DNA constructs into seminal vesicle tissues or annex tissues can be performed to treat a disease, such as a cancer for example, or to modulate the physiology of different targeted cell groups in these tissues.

An expression vector or a cell transformed with a vector comprising a seminal vesicle tissue specific promoter as described in the present invention, and regulating the expression of a gene of interest, can be directly integrated into the seminal vesicle cells of a patient in needs. For example, but without limiting the scope of the present invention, integration of a cell growth inhibitor can be secreted directly by cells that are up-regulated, and being hyperproliferative. Hyperproliferation disorder is one cell behavior that can be treated in this manner. In this case, if a part of the expression vectors are liberated into the organism, the tissue specificity of the promoters used restricted the expression of the contiguous gene in the seminal vesicle and annexes only, avoiding uncontrolled expression elsewhere in the treated organisms or patient.

The problem of producing active recombinant proteins with post-translational modifications may be overcome in accordance with the present invention, by the use of the pig genital tract as a tissue of expression. Semen is readily collected, available in large quantities in several animal species and well characterized biochemically. Furthermore, several proteins are present at high concentrations in this body fluid.

In accordance with the present invention, the seminal gland of transgenic pigs is used as a production system of recombinant protein.

The present invention solves such problems by providing new efficient means of producing large quantities of recombinant protein products in the semen of transgenically altered animal.

It has been surprisingly observed by the present inventors that very complex proteins, as for example but not limited to, dimeric proteins, can be produced in the seminal vesicle and sexual accessory glands of pigs.

More precisely, this invention relates to processes, DNA sequences, compositions of matter and transgenic pigs for the production of recombinant proteins. More specifically, this invention relates to the transgenic incorporation of one or more copies of a construct comprised of a genital tract-specific protein promoter or any promoter sequence specifically activated in genital tract tissue, operatively linked to a DNA sequence coding for a desired recombinant protein or combination of proteins through a DNA sequence coding for a signal peptide that permits the secretion and maturation of the desired recombinant protein in the genital tract tissue. The gene construct is incorporated into pig embryos or stem cells or adult cells used for cloning and the recombinant protein products are subsequently expressed and secreted into or along with the semen of the transgenic pig.

Any animal may be usefully employed in this invention. Preferably, animal that produce large volumes of semen and have frequent ejaculating periods are preferred.

The protein products that may be produced by the processes of this invention include, for example, mono- or antibodies or derivatives thereof, immunoglobulins, cytokines, coagulation factors, tissue plasminogen activator. GM-CSF, erythropoietin, thrombopoietin, alpha-1 antitrypsin, animal growth hormones, cell surface proteins, insulin, interferons, lipases, antiviral protein, antibacterial protein, bacteriocins, peptide hormones, lipocortins and other recombinant protein products. Particular proteins that can be recombinantly produced with the present production system are dimeric proteins, such as, without limiting it to, LH, TSH, and FSH, which are heterodimeric proteins.

The desired recombinant protein may be produced as a fused protein containing amino acids in addition to those of the desired or native protein. For example, the desired recombinant protein of this invention may be produced as part of a larger recombinant protein in order to stabilize the desired protein or to facilitate its purification from semen. The fusion is then broken and the desired protein isolated. The desired recombinant protein may alternatively be produced as a fragment or derivative of native protein or it may be produced having an amino acid sequence similar to the native protein. Each of these alternatives is readily produced by merely choosing the correct DNA sequence.

The above-described transgene may be prepared using methods well known in the art. The transgene for the current invention is configured preferentially for activate then express a desired recombinant protein once integrated in a pig genome. For example, various ligation techniques employing conventional linkers, restriction sites etc., may be used to good effect. Preferably, the expression system of the present invention are prepared as part of larger plasmids or other vectors such as cosmids, BACs and PACs. Such preparation allow for the cloning and selection of the correct constructions in an efficient manner as it is well known in the art. Preferentially, the transgene of this invention can be located between convenient restriction sites on a plasmid so that they can be easily isolated from the remaining plasmid sequences for incorporation into the desired animal.

After such isolation and purification, the expression systems or constructs of this invention are added to the gene pool of the pig which is to be transgenically altered. One or several copies of the construct may be incorporated into the genome of a pig embryo by standard or novel transgenic techniques. The pig has been shown to produce up to 500 ml of semen/ejaculation. This appears to be an animal of choice for the production of recombinant proteins of interest in the semen.

One technique for transgenically altering an animal is to microinject the transgene into the pronuclei of the fertilized egg(s) to cause one or more copies of the transgene to be retained in the cells of the developing pig animals. Usually, transgenic pigs contain at least one copy of the cloned transgene in both somatic and germinal tissues and transmit the gene through the germ line to the next generation. The progeny of the transgenically manipulated embryos may be tested for the presence of the construct by Southern blot analysis of a biopsy of tissue or amplification of a transgene sequence by polymerase chain reaction technique. If one or more copies of the exogenous cloned construct remains stably integrated into the genome of such transgenic embryos, it is possible to establish permanent transgenic animal lines carrying the transgenically added construct.

According to another embodiment of the present invention, different proteins of subunits of hetero- as well as homo-dimeric proteins, can be secreted together in the same pig's semen after co-introduction of transgenes producing different and/or independent protein subunits. For example, the promoter two co-integrated transgenes encoding for two independent proteins or subunits, can differ or be the same as long as they remain specific to the seminal vesicle or male accessory sex glands

The litters of transgenically altered animals may be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those progeny found to contain at least one copy of the construct in their genome are grown to maturity. The male species of these progeny will produce the desired protein along with their semen. Alternatively, the transgenic animal may be bred to produce other transgenic progeny useful in producing the desired proteins in their semen.

The ability to introduce and express exogenous genetic information in the context of a developing organism allows the investigation of the mechanisms regulating specific gene expression and the role of such expression in normal development and pathological states. Since the first success at producing transgenic mice using microinjection of eggs with cloned genes (Gordon et al., 1980, Proc. Natl. Acad. Sci. USA 77:7380-7384), numerous investigators have employed this technique to study various developmental processes. The use of transgenic mice to study gene regulation has steadily increased, and is considered as an important in evaluating DNA constructs before using them to produce transgenic farm animals. However, time has proven that mouse is in only a model and results obtained in mice cannot be systematically correlated with other species, particularly when the physiology of the same organs is significantly different. It will be recognized by someone skilled in the art that the physiology of the seminal vesicle and accessory glands of pigs are different from those of the mouse.

The biochemical characterization of fluid semen and proteins of the semen of male mammals is still incomplete. However, data available at the moment regarding this aspect are in sufficient quantities to say that qualitative and quantitative biochemical composition between different species in male mammals, are quite different.

The transgenic animal bioreactor industry has focused primarily on directing expression of their products to the milk, though at least one other organization has explored the possibility of isolating products from blood or urine (Swanson et al., 1992, Bio/Technology 10:557-559). The present invention provides an alternative (i.e. using the genital tract and sexual accessory glands as a bioreactor) that has the same advantage of mammary gland bioreactors: straightforward, noninvasive collection of the product.

The present invention will be more readily understood by referring to the following example which is given to illustrate the invention rather than to limit its scope.

### EXAMPLE I

### Preparation of Expression Vectors

For the assays described below, the expression vectors (or transgene) basic elements are a promoter fragment from either porcine PSP-I (8 kb=“G long” promoter, 7 kb=“G” promoter)(SEQ ID NO:1), PSP-II (“J3” promoter, 13 136 bp)(SEQ ID NO:2), or AQN-3 (15 572 bp)(SEQ ID NO:3) genes.

The protein encoded by the vector is an artificially fused peptide in which both chains of the normally dimeric porcine FSH are produced as a single protein. This is identified in constructs as “FSH_{f}”.

### EXAMPLE II

### In vitro Transfections and Biological Assays

The promoter's capacity to activate transcription in cells transfected in vitro was evaluated by the presence of FSH-like biological activity in the cells medium 48 hours after transfection.

This activity was evaluated by the capacity of conditioned medium to cause cumulus expansion in a qualitative assay using bovine and porcine ova, with appropriate positive and negative controls. In order to rapidly assess the porcine promoters PSP-I and PSP-II we cloned, we constructed expression vectors that encoded porcine follicle-stimulating hormone (FSH) under control of a large genomic fragment from either PSP-I or PSP-II promoters.

These expression vectors were tested in parallel with one that used the CMV promoter and the same coding sequence as a control. Primary cultures of pig seminal vesicle epithelium cells were used as a model for seminal vesicles specific tissue and pig granulosa cells as non-specific tissue for transfections with Lipofectamine 2000™.

The presence of FSH_{f} in the culture medium of transfected cells was verified with a qualitative biological activity assay. In this assay, bovine or porcine ova still surrounded by their cumulus were harvested and placed in 6-well cell culture dishes with maturation medium in a CO₂ incubator at the appropriate temperature. The ova were exposed to conditioned medium collected 48 hrs post-transfection. In the presence of as little as 5 ng/ml of pure FSH, the cumulus shows clearly visible expansion. However, a concentration of 500 ng/ml was used as positive control. Conditioned medium from transfected cells was added at 5% and 10% final concentration in the ova maturation medium. Medium from untransfected cells was used as negative control.

TABLE 1
Presence or absence of cumulus expansion in presence
of conditioned media from cells transfected with
different expression vectors

Cells transfected

| | Porcine | Porcine seminal |
|---|---|---|
| Promoter used | Granulosa | vesicles epithelium |
| CMV | + | + |
| G long (PSP-I, 8 kb) | − | + |
| J3 (PSP-II, 7 kb) | − | + |

### EXAMPLE III

### In vivo Transfections in Mice

A 8 kb fragment of porcine PSP-I promoter was tested by in vivo transfection in mice, either through IV injection or directly in seminal vesicles with DNA and ExGen™500. Promoter activity was verified by the presence of FSH_{f} in seminal fluid both by cumulus expansion and RIA assay.”

The J3P8-FSH_{f} DNA construct was used for in vivo transfection assays in mice with the ExGen™500 reagent (PEI) from Fermentas. Transfection was performed either by intravenous injection (IV) of the DNA/PEI mixture in the tail vein or by direct injection into the lumen of the seminal vesicles (SV). Seminal fluid was collected 24 hrs after treatment and the amount of porcine FSH was evaluated with a commercial RIA assay. The negative control (Neg ctrl) was seminal fluid from a mouse injected with PEI alone directly in SV.

It appears that IV injection results in DNA delivery as well as direct SV injection. Also, tissues from mice IV injected with the same construct are used to show by RT-PCR that the porcine promoter J3P8 is tissue-specific in mouse.

### EXAMPLE IV

### Production of Transgenic Mice with Semen Vesicle Promoters

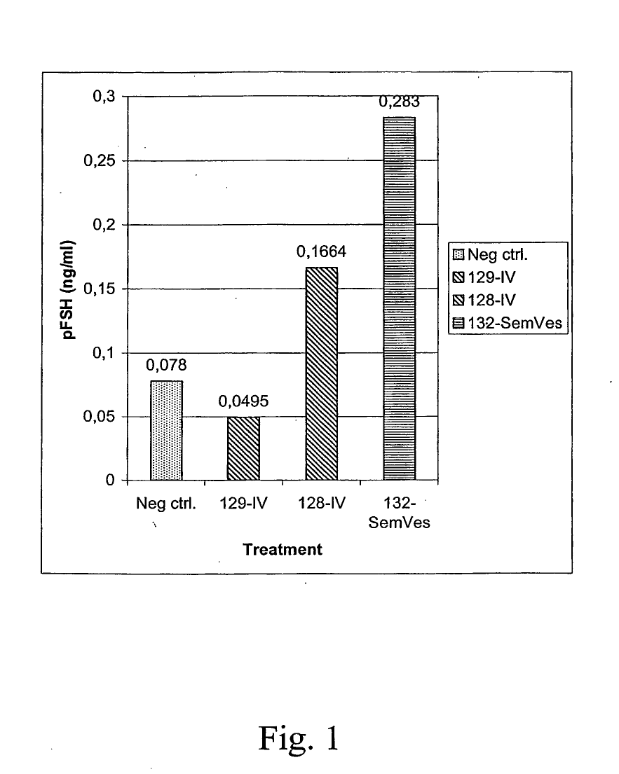

A male founder was produced with the “G long-FSH” construct (PSP-I promoter). At 7 weeks of age the subject became sexually mature and the presence of FSH in his seminal fluid was tested from vaginal plugs of females he mated. pFSH RIA results in mouse seminal fluid after in vivo transfection are illustrates in FIG. 1.

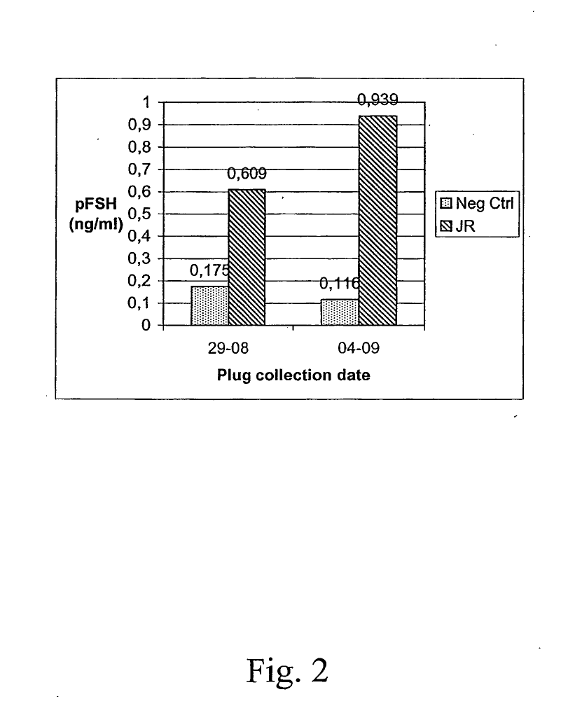

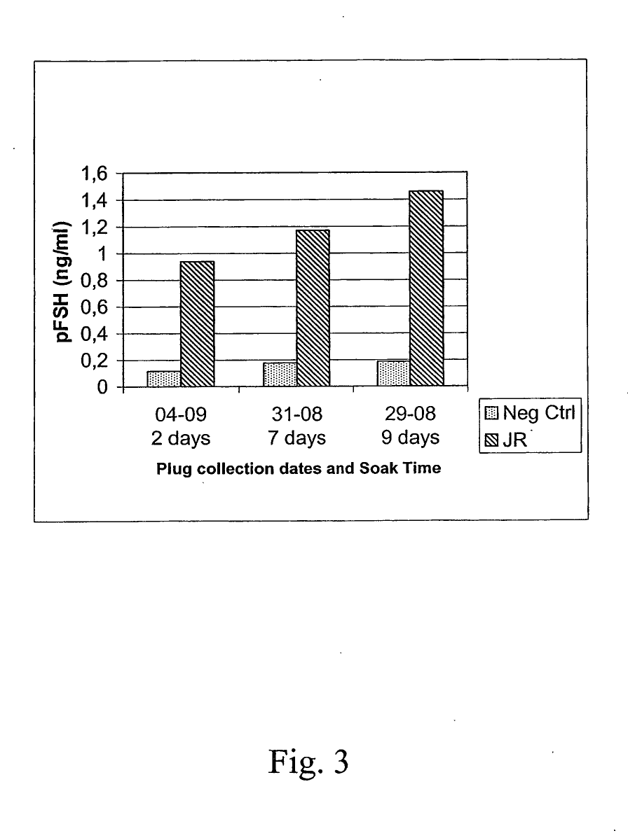

The “G long-FSH” construct was microinjected in mouse embryos pronuclei. A male transgenic founder was identified and labeled “JR”. Upon reaching sexual maturity (7 weeks of age) the animal was mated and vaginal plugs were collected. The plugs were sliced and left to soak in a physiological buffer for various lengths of time, depending on the collection date and the day when the RIA assay was performed. Production seemed to increase after a week (FIG. 2), but the amount of pFSH present in the buffer seems to depend more on soak duration which seems to lead to increased pFSH liberation into the buffer (FIG. 3).

### EXAMPLE V

### Production of Recombinant hFSH and pFSH in Mice and Pigs

Material and Methods

Tissue-specific Promoters Cloning

Primers were designed from published porcine PSP-I cDNA sequence and used in a PCR reaction using pig genomic DNA template and Expand High Fidelity enzyme mix. The resulting 3.3 kb fragment was cloned in pGEM-T_{easy} and the corresponding sequence indicated it contained the first part of PSP-I transcribed region from the end of exon 1 to the beginning of exon 3. A 1.3 kb EcoR I-BamH I fragment containing the end of exon 1 and the first part of intron 1 was purified from this plasmid. This fragment was further digested with Pst I to generate three unequal fragments measuring 120 bp, 532 bp and 696 bp which were used as a mix to generate the probes to screen the genomic library. The 532 bp fragment sequence comes entirely from the start of intron 1 which shows very high homology between pig spermadhesins PSP-I, PSP-II and AQN-3. The 696 bp fragment 3′ end is more specific to PSP-I than to other known spermadhesins sequences. The mix of all three fragments should thus generate a probe which can hybridize to more than one spermadhesin genomic clone but nonetheless yield a stronger signal with PSP-I clones. The ³²P-labelled probe was generated with the T7 Quick Prime kit (Pharmacia).

Library Screening:

For the first screening round, a CLONTECH porcine λ genomic library was plated on ten 23 cm×23 cm plates at 10⁵ pfus per plate, for a total of 10⁶ pfus. Duplicate plaque lifts of each plate were made with 20 cm×20 cm positively charged nylon membranes (Boehringer-Mannheim) and probed with 2.0×10⁴ cpm/cm² using Church's Buffer for prehybridization and hybridization. Nine positive matching duplicate signals of varying intensities were identified. For each clone, a 1 cm² plug centered on the signal was cut out and subjected to a second screening round. Only five of the twelve candidates gave strong enough signals to be retained after the second round. These were named G, J1, J2, J3 and I, from the plates on which they were identified during the first screening round. Isolated plaques from the second round were picked out and subjected to a third round to verify each clone had been brought to purity.

Characterization and Identification of λ Clones

Relative sizes of the genomic sequences upstream and downstream of the transcription start signal (TSS) were evaluated by PCR reactions using primers based on the λ arms sequences next to the cloning site and a sequence at the beginning of PSP-I intron 1 that was perfectly conserved in PSP-II and AQN-3 corresponding regions. The three clones which had regulatory regions equal to or larger than 10 kb, G, I and J3, were retained for further study. Their regulatory and transcribed region sections were independently sub-cloned by PCR into either pGEM-T_{easy} or pCR-XL-TOPO according to their size and sequenced. The transcribed regions provided data to identify which pig spermadhesin gene each clone corresponded to.

GP8 and J3P8 Promoters Plasmids

Clone G was identified as PSP-I and its TSS upstream region measured 10 kb. Clone J3 was identified as PSP-II and its TSS upstream region measured approximately 15.5 kb. The PCR fragments containing the complete TSS upstream regions from λ clones G and J3 DNA had been cloned in the Invitrogen™ TA-cloning vector pCR-XL-TOPO to generate plasmids pTOPO-GP and pTOPO-J3P. These plasmids were treated with the New England Biolabs kit GSP-1 to generate libraries of sub-clone plasmids containing a randomly inserted transposon of known sequence in each sub-clone.

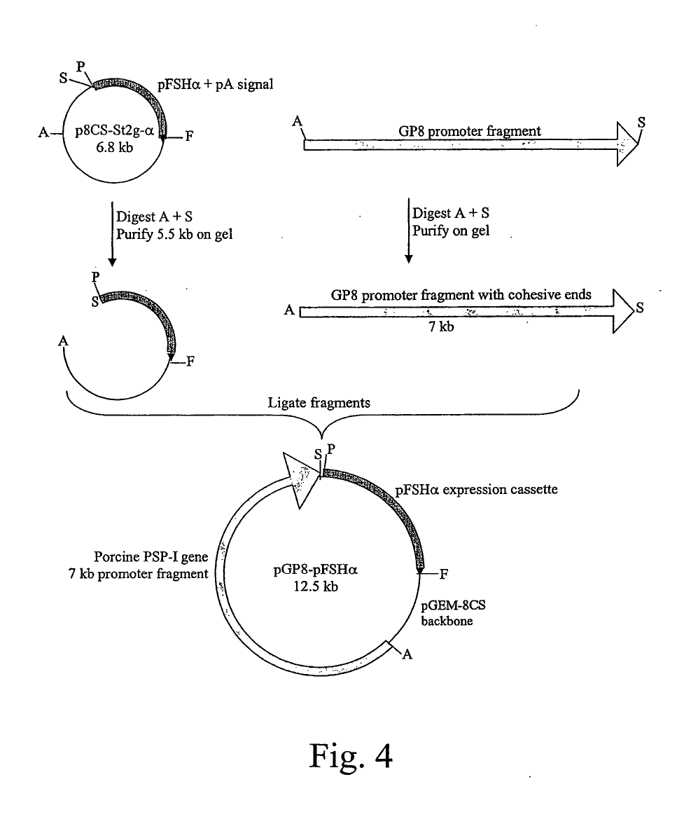

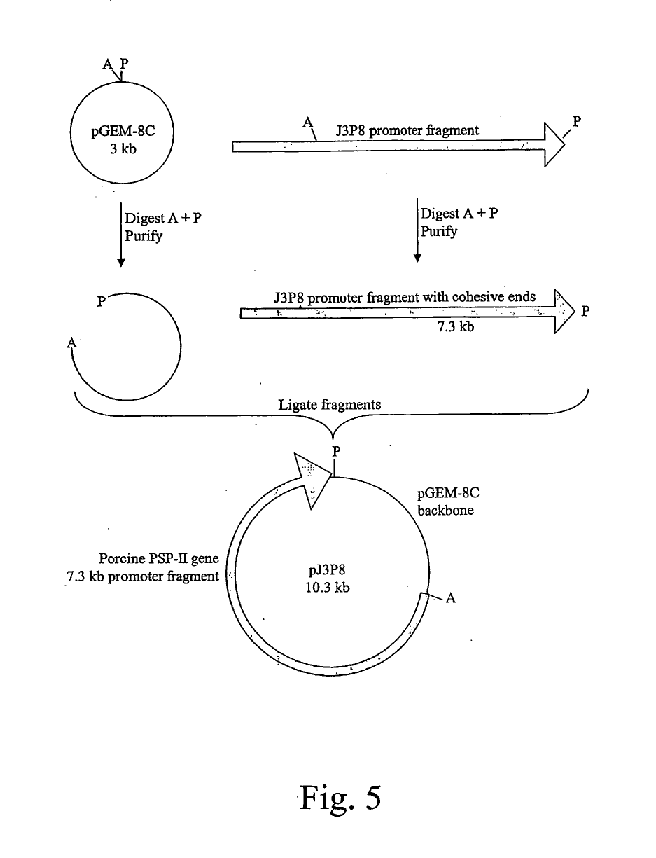

To identify the transposon relative orientation and approximate insertion point in a number of sub-clones, we ran PCR analyses with primers based on the ends of the original GP and J3P PCR clones and on those of the transposon used in the GSP-1 reaction. The strategies followed in preparing plasmids that contain shorter promoter fragments are shown in FIGS. 4 and 5. Enough sub-clones from each library were analyzed to identify one which had an insertion point positioned approximately between 7 kb and 8 kb upstream of the TSS. Promoter fragments called GP8 and J3P8 were generated by PCR from these selected sub-clones with a forward primer based on the appropriate end of the transposon and a reverse primer targeted to the genes 5′-UTR sequence. This reverse primer included the first two bases of the initiator ATG codon. Both forward and reverse primers had extended 5′ ends containing rare cutter restriction enzyme sites to allow subsequent cloning of each promoter fragment into a modified pGEM plasmid called pGEM-8C which had Asc I, Sda I, Pme I, Pac I and Fse I extra sites engineered into the MCS. The GP8 promoter fragment (FIG. 4) was cloned into p8CS-ST2G-α with Asc I and Sda I cohesive ends while J3P8 (FIG. 5) was cloned with Asc I and Pac I cohesive ends. This resulted in vectors pGP8 and pJ3P8 (FIGS. 4 and 5).

Cloning pFSHα and pFSHβ and in vitro Expression

PCR primers that amplified porcine genomic fragments containing the complete coding sequences for pFSHα and pFSHβ were used. These fragments were cloned into the pCR3.1-Uni TA-cloning vector (Invitrogen), which allows unidirectional cloning and provides the necessary elements for eukaryotic expression: strong promoter and polyadenylation signal (polyA). The pCR3.1-FSHβ vector was further modified by deleting a 1 kb central fragment from the 1.5 kb intron in order to generate a mini-gene for FSHβ called FSHβΔ.

Vectors pCR3.1-FSHα and pCR3.1-FSHβΔ were transfected in pig granulosa cells and the medium was collected after 48 hours. The presence of biologically active dimerized pFSH was verified with a in vitro biological assay based on cumulus expansion of bovine oocytes. (Choi et al., Teriogenology, 2001., 56:661-670)

Constructs for pFSH Transgenes

DNA constructs in which the pFSH sub-units genomic coding sequences were joined to pig spermadhesins promoter clones GP8 and J3P8 were prepared. Each sub-unit sequence was amplified with PCR primers designed to contain restriction sites Pac I and Fse I for cloning into pJ3P8. The primers were designed to amplify from the pCR3.1-pFSH vectors fragments that included the pFSH sub-unit sequence joined to the vector polyA signal. After ligation into the appropriately treated pJ3P8 we obtained vectors pJ3P8-pFSHα and pJ3P8-pFSHβΔ in which porcine tissue-specific promoter J3P8 was joined to pFSH sub-units followed by a strong polyA signal.

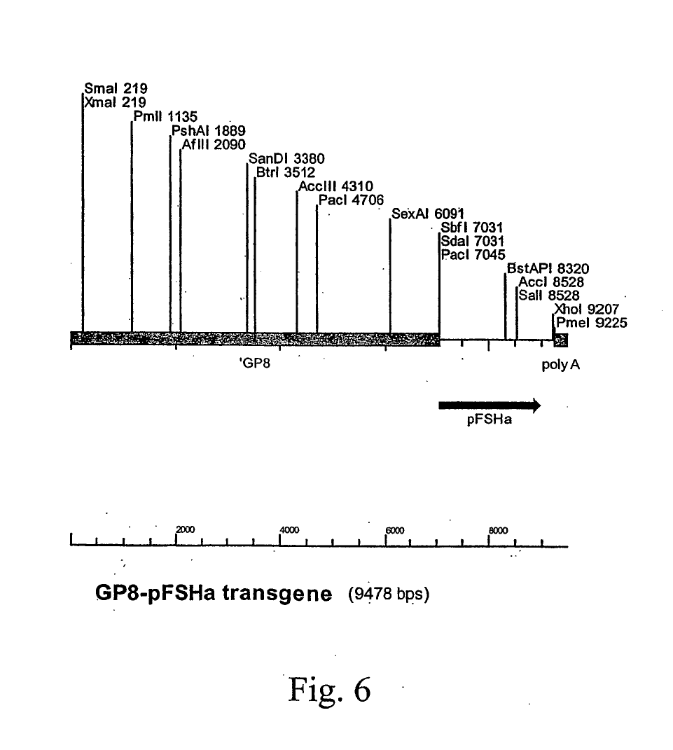

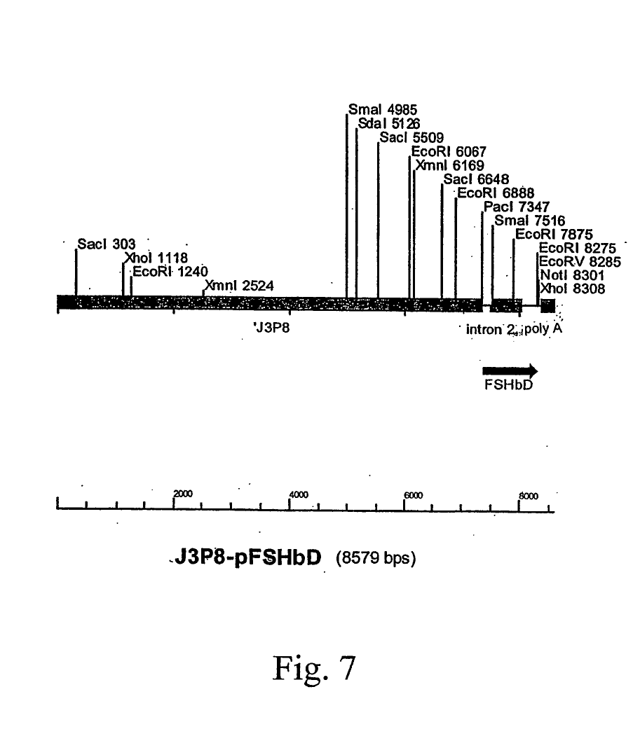

For the GP8 promoter constructs, we first created a preliminary plasmid called p8CS-St2G. This plasmid is based on the previously described pGEM-8C into which we introduced two stuffer DNA fragments between restriction sites Asc I and Sda I for stuffer 1, and between sites Pac I and Fse I for stuffer 2. We then replaced stuffer 2 with the PCR fragment containing pFSHα joined to a polyA signal by sequential treatment of both plasmid and fragment with enzymes Pac I and Fse I, gel purification and ligation to generate plasmid p8CS-St2G-α. Subsequently, we replaced stuffer 1 with the GP8 promoter fragment by sequential treatment of both plasmid and fragment with enzymes Asc I and Sda I, gel purification and ligation to generate plasmid pGP8-pFSHα (FIG. 4). We followed a similar procedure to generate plasmid pGP8-pFSHβΔ. The restriction maps are illustrated in FIGS. 6 and 7.

Microinjection of the Gene Construct into Mouse and Pig Embryos

Production of transgenic mice by introduction of DNA constructs into the pronucleus of fertilized oocytes was performed as per established protocols (Hogan et al. 1994). Briefly, donor females (strain B6C/3F1 Charles River, Wilmington Mass.) were superovulated using Pregnant Mare Serum Gonadtrophins (PMSG, Vetrepharm, London, ON, Canada) and human Chorionic Gonaditropin (hCG, InterVet, Whitby, ON), mated to fertile males and sacrificed the following day. Embryos at the pronuclear stage were then recovered from the oviducts and one of the nuclei microinjected with the DNA construct. The injected embryos were reimplanted into pseudopregnant recipient females (strain CD1, Charles River, Wilmington Mass.), which have been mated with vasectomized males.

Porcine embryos were produced using cycling Landrace-Yorkshire X Duroc gilts with an approximate weight and age of 100 kg and 6 months, respectively. These embryo donors were synchronized and superovulated by the following steps: 17 days of oral Altrenogest™ treatment (Regumate, Hoechst Canada Inc); one day following the end of the altrenogest treatment—injection of 1200 to 1400 IU of PMSG (Vetrepharm, London, ON, Canada); injection of 1000 IU hCG (InterVet, Whitby, ON) 78 hours following the PMSG injection; artificial insemination, 24 and 36 hours following the hCG injection; embryos isolated from the oviducts, 52 to 57 hours following the hCG injection. The collection and transfer of embryos was performed by mid-ventral laparotomy with the gilt under general anesthesia as approved by the institutional animal care and use committee of the Université Laval. Embryos were flushed from the oviducts using two 15 ml volumes of warmed (37° C.) Phosphate Buffered Saline (PBS) with 0.4% (w/v) BSA (Sigma, Mississauga,On, Canada) and 50 mg/ml gentamycin (Sigma, Mississauga, On, Canada). To allow for the visualization of the pronuclei, embryos were then centrifuged at 15,000×g for 10 min (Wall et al., 1985). Embryos at the pronuclear stage were identified and one of the nuclei microinjected with the DNA solution at concentration of 4 ng/μl. Surviving embryos were then reimplanted into the oviduct of a recipient female. Recipients were prepared in a similar manner as the donors, however insemination was not performed. A mid-ventral laparotomy was performed on the recipients, and those which were identified by ovarian morphology as having ovulated, had injected embryos inserted into their oviducts htrough a small canula and a small volume of phosphate buffered saline (PBS).

Offspring resulting from the recipient mothers were evaluated by PCR and Southern Blot for the presence of the transgene to identify potential transgenic founder animals.

Identification of Transgenic Animals

Mouse tail tissue was collected and immediately digested with pronase-K (0.4 mg/ml) in 50 mM Tris-HCl (pH 7.4), 1 mM EDTA and 1% sodium dodecyl sulfate (SDS) overnight at 55° C. Pig tail and ear tissues collected and frozen for transport to the laboratory where the tissue was digested as for the mouse tails. High molecular weight DNA was isolated by phenol-chloroform extraction and precipitated in 95% ethanol. Isolated genomic DNA was washed twice in 70% ethanol. The DNA pellet then dissolved in 10 mM Tris-HCl (pH 8.0),1 mM EDTA and stored at 4° C. until time of use.

PCR identification of transgenic animals was performed using primers designed to specifically amplify a DNA fragment of predetermined size from each transgene. Amplification products were separated by electrophoresis on a 1.0% agarose gel and the presence of an anticipated band indicating a transgenic animal.

Southern analysis was performed using established methods (Sambrook et al., 1989). Briefly, genomic DNA was digested overnight with Pst I restriction endonuclease under appropriate conditions. DNA fragments were separated by electrophoresis on an 0.8% agarose gel and transferred to nylon membranes (Roche, Laval, QC Canada). Equal loading of lanes and the rate of DNA digestion were evaluated by ethidium bromide staining of the agarose gel prior to transfer. The probes used for these studies consisted of p³² labelled DNA fragment specific to each transgene prepared by random priming.

Prehybridization and hybridization was carried out with the indicated probes in a standard hybridization solution at 42° C. Membranes were washed twice for 15 min at 55-65° C. in 0.5% SDS, 0.1% SDS. Bound probe was detected using a Phosphoimager (Molecular Dynamics- Amersham Biosciences, Sunnnyvale, Calif. USA).

Detection of Recombinant pFSH by Radioirnmunoassay (RIA)

The concentration of pFSH in mouse vaginal plug extracts pig seminal fluid was determined by RIA. Vaginal plug extracts were produced by placing transgenic and non-transgenic males with PMSG-hCG synchronized females overnight and collecting the vaginal plug the next morning. The vaginal plug was placed in physiological buffer so that the FSH within the plug could diffuse into solution.

Boar semen was collected by the gloved-hand technique and seminal fluid separated from the sperm by centrifugation at 800 g. Samples were placed at −20 Celsius until the time of assay. Concentrations of pFSH were determined using a RIA kit (Biocode, Belgium) or a double anti-body RIA protocol as described by the supplier of the primary pFSH antibody (Biogenesis, Brentwood NH USA).

RESULTS

TABLE 1
Detection of porcine FSH in mouse semen

| | Vaginal Plug Extracts |
|---|---|
| Transgenic Mouse Line | pFSH (ng/ml) |
| J3P8-pFSHα + GP8-pFSHβΔ | 8.43 |
| GP8-pFSHα + J3P8-pFSHβΔ | 167.45 |

TABLE 2
Detection of recombinant porcine FSH in pig semen

| Transgenic Founder Boar ID | Seminal Fluid |
|---|---|
| (GP8-pFSHα + J3P8-pFSHβΔ) | pFSH (μg/ml) |
| CO4P | 24.00 |
| DO1P | 58.81 |
| F02P | 7.4 |
| H10P | 7.5 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. An isolated DNA sequence which substantially regulates expression of a heterologous gene, wherein said isolated DNA sequence is selected from the group consisting of SEQ ID NO:1, SEQ ID NO:2; SEQ ID NO:3, SEQ ID NO:4 and a functional fragment thereof, or a DNA sequence having at least 80 percent of homology with said isolated DNA sequence.

2. The isolated DNA sequence of claim 1, wherein said expression of heterologous gene is performed in a mammal seminal vesicle and accessory tissues thereof.

3. The isolated DNA sequence of claim 2, wherein said mammal seminal vesicle and accessory tissues thereof is of a human, a porcine, a bovine, a primate, a caprine, an ovine, an equine, a murine, a canine, or a feline origine.

4. A DNA construct comprising a DNA sequence encoding a heterologous RNA or protein operably linked to an isolated promoter having an isolated DNA sequence as defined in claim 1, or a DNA sequence having at least 80 percent of homology with said isolated DNA sequence.

5. A mammalian cell having a genome which comprises a DNA construct as defined in claim 4.

6. The mammalian cell of claim 5, wherein said cell is from a seminal vesicle or accessory tissues thereof.

7. The mammalian cell of claim 5, wherein said mammalian cell is a human, a porcine, a bovine, a primate, a caprine, an ovine, an equine, a murine, a canine or a feline cell.

8. A DNA construct of claim 4, wherein said RNA is an anti-mRNA or a ribosyme.

9. A non-human genetically modified mammal whose seminal vesicle or accessory tissues thereof contain a DNA sequence encoding a heterologous protein operably linked to a DNA construct as defined in claim 3, a fragment thereof or a DNA sequence having at least 80 percent of homology with said isolated DNA sequence thereof, wherein said non-human genetically modified mammal is naturally or artificially physiologically inducible to express said heterologous protein in seminal vesicle cells or accessory tissues thereof, whereby the heterologous protein is detectable in semen or seminal secretion produced by said transgenic or chimeric mammal.

10. The non-human genetically modified mammal of claim 9, wherein expression of said heterologous protein is artificially induced by hormone administration to said non-human genetically modified mammal.

11. The non-human genetically modified mammal of claim 9, wherein said mammal is a porcine, a bovine, a primate, a caprine, an ovine, an equine, a murine, a canine or a feline.

12. A mammal whose seminal vesicle cells or accessory tissue cells thereof have a genome comprising a DNA sequence encoding a heterologous protein operably linked to an isolated DNA sequence as defined in claim 1, or a DNA sequence having at least 80 percent of homology with said isolated DNA sequence, wherein said seminal vesicle cells or accessory tissue cells thereof are naturally or artificially physiologically inducible to express said heterologous protein whereby the heterologous protein is detectable in semen or seminal secretion produced by said mammal.

13. A method for producing a recombinant protein comprising the step of:
a) introducing into a cell a DNA construct as defined in claim 4 to form a genetically transformed cell; and
b) naturally or artificially inducing said genetically transformed cell of step a) to express said heterologous protein.

14. The method of claim 13, wherein said cell is a cell from a seminal vesicle or an accessory tissue thereof.

15. The method of claim 13, wherein said genetically transformed cell of step b) is further cultured in vitro or present in a mammal.

16. The method of claim 13, wherein said heterologous protein is produced in a culture medium in which said genetically transformed cell is cultured or in the semen of said mammal.

17. Use of an isolated DNA sequence as defined in claim 1 for regulating the expression of an heterologous RNA or protein in a mammal.

18. A method for producing recombinant protein in semen of pigs comprising the steps of:
a) introducing in the genome of a pig cell at least one transgene comprising a signal sequence, a DNA sequence encoding for a protein of interest or a part thereof operably linked to a promoter allowing the expression of said DNA sequence exclusively in seminal vesicle or male sexual accessory glands to produce a transgenic pig cell, said transgene being integrated in said genome in manner said promoter is activated in a sexually matured pig and said DNA sequence is expressed; and
b) developing said transgenic pig cell into sexually mature transgenic pig,
wherein the semen of said sexually mature transgenic pig contains said recombinant protein.

19. The method of claim 18, wherein said pig cell is an oocyte, a zygote, a sperm or a somatic cell.

20. The method of claim 18, wherein said protein of interest is an homo- or an heterodimeric protein.

21. The method of claim 18, wherein said protein of interest is composed of at least one subunit of an homo- or heterodimeric protein.

22. The method of claim 21, wherein said protein of interest is composed of at least one functional subunit of follicle stimulating hormone.

23. The method of claim 18, wherein said protein of interest is erythropoietin.

24. The method of claim 22, wherein said subunit is an alpha- or a beta-subunit.

25. The method of claim 18, wherein said promoter is selected from the group of p25, p34, kallikreins, PSA, SBP-C, GP8, J3P8, PsP-1, PSP-2, PSP-3, or secretory protein IV promoters.

26. The method of claim 18, wherein said promoter is selected from the group of SEQ ID NO: 1, SEQ ID NO:2, SEQ ID NO:3, and SEQ ID NO:4.

27. The method of claim 18, wherein said genome of pig cell is genetically transformed with one transgene encoding for a part or subunit of a protein, and another transgene encoding for another part or subunit of the same protein.

28. A transgenic pig having genome genetically modified with at least one transgene comprising a signal sequence, a DNA sequence encoding for a protein of interest or a part thereof operably linked to a promoter allowing the expression of said DNA sequence in seminal vesicle or male sexual accessory glands.
